(19) United States
(12) Patent Application Publication
(10) Pub. No.: US 2018/0313846 A1
(43) Pub. Date: Nov. 1, 2018

(54) BLOOD TEST TO PREDICT ENDURANCE ATHLETIC PERFORMANCE

(71) Applicant: Maine Medical Center, Scarborough, ME (US)

(72) Inventor: Volkhard Lindner, South Portland, ME (US)

(21) Appl. No.: 15/944,585

(22) Filed: Apr. 3, 2018

Related U.S. Application Data
(60) Provisional application No. 62/480,887, filed on Apr. 3, 2017.

(51) Int. Cl. G01N 33/68; G01N 33/96

(52) U.S. Cl. CPC G01N 33/68; G01N 33/96

(57) ABSTRACT

The invention features compositions and methods for predicting endurance athletic performance.

### CROSS-REFERENCE TO RELATED APPLICATIONS

The present application claims the benefit under 35 U.S.C. § 119(e) of U.S. Provisional Patent Application No. 62/480,887, filed Apr. 3, 2017, the entire contents of which are hereby incorporated by reference.

### FIELD OF THE INVENTION

This invention relates generally to the field of athletic performance.

### SEQUENCE LISTING

Incorporated by reference in its entirety is a computer-readable nucleotide/amino acid sequence listing submitted concurrently herewith and identified as follows: 8,192 byte text file named “48420_516001WO_Sequence_Listing_ST25.txt” created on Apr. 3, 2018.

### BACKGROUND OF THE INVENTION

Collagen Triple Helix Repeat Containing 1 (CTHRC1) is a circulating factor expressed at sites of tissue injury and remodeling. However, prior to the invention described herein, the role of CTHRC1 function in athletic performance had not been identified. As such, there is a pressing need to identify the role of CTHRC1 in athletic performance.

### SUMMARY OF THE INVENTION

The invention is based, at least in part, on the surprising discovery that Collagen Triple Helix Repeat Containing 1 (CTHRC1) levels predict endurance athletic performance. Methods of predicting endurance athletic performance in a subject are carried out by providing a test sample from the subject; determining a level of CTHRC1 in the test sample from the subject; and comparing the level of CTHRC1 in the subject to a control level, wherein a higher level of CTHRC1 in the test sample relative to the control sample indicates that the subject has high endurance. In one aspect, the level of CTHRC1 is determined using an enzyme-linked immunosorbent assay (ELISA). In some cases, the subject with high endurance has better (i.e., greater) physical performance under physically stressful conditions as compared to a subject with low or normal endurance. For example, the subject with high endurance has a physical performance that is at least 5% greater, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater as compared to a subject with low or normal endurance.

In various embodiments, the subject has high endurance if the level of CTHRC1 is at least about 5%, 10%, 15%, 20%, 25%, 30%, 40%, 45%, 50%, 55%, 60%, 65%, 70%, 75%, 80%, 90%, 5-50%, 50-75%, 1-fold, 2-fold, 3-fold, 4-fold, or 5-fold higher in said test sample compared to a normal control.

In some embodiments, the level of CTHRC1 is determined at least about 1, 2, 3, 4, 5, 6, 7, 8, 9, 10 or more times and/or at least once every 1, 2, 3, or 4 weeks; at least once every 1, 2, 3, 4, 5, or 6 weeks; or at least once every 1, 2, 3, 4, or 5 years.

In embodiments, the test sample comprises a blood sample, e.g., a plasma sample. In one aspect, CTHRC1 levels are examined at least once per month in the subject, e.g, at least twice per month, at least once per week, at least twice per week, at least three times per week, at least four times per week, at least once per day, at least twice per day, or at least once per hour. The subject is preferably a mammal. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Provided herein are CTHRC1 polypeptide agonists and antagonists.

The term “agonist” as used herein, refers to any molecule which enhances the biological activity of its target molecule.

As used herein, the terms “antagonist” and “inhibitor” are used interchangeably to refer to any molecule that counteracts or inhibits, decreases, or suppresses the biological activity of its target molecule. Suitable CTHRC1 polypeptide antagonists include soluble receptors (e.g., soluble CTHRC1 receptor), peptide inhibitors, small molecule inhibitors, ligand fusions, and antibodies.

The term “receptor antagonist,” as used herein, refers to an agent that is capable of specifically binding and inhibiting signaling through a receptor to fully block or detectably inhibit a response mediated by the receptor.

The agonists or antagonists may include but are not limited to nucleic acids, peptides, antibodies, or small molecules that bind to their specified target or the target's natural ligand and modulate the biological activity.

The present invention provides diagnostic binding agent conjugates. In some embodiments, the binding agent comprises a nucleic acid molecule, such as a sequence that is complementary to mRNA or cDNA produced from mRNA that encodes CTHRC1. In some aspects, the present subject matter provides a composition utilizing a binding agent, wherein the binding agent is attached to a solid support, (e.g., a strip, a polymer, a bead, a nanoparticle, a plate such as a multiwell plate, or an array such as a microarray). In embodiments relating to the use of a nucleic acid probe attached to a solid support (such as a microarray), nucleic acid in a test sample may be amplified (e.g., using PCR) before or after the nucleic acid to be measured is hybridized with the probe. Various embodiments comprise reverse transcription polymerase chain reaction (RT-PCR) to detect mRNA levels (e.g., to determine the level of CTHRC1). In some embodiments involving a probe on a solid support, the mRNA (or a portion thereof) in a test sample is converted to cDNA or partial cDNA and then the cDNA or partial cDNA is hybridized to a probe (e.g., on a microarray), hybridized to a probe and then amplified, or amplified and then hybridized to a probe. In some examples, a strip may be a nucleic acid-probe coated porous or non-porous solid support strip comprising linking a nucleic acid probe to a carrier to prepare a conjugate and immobilizing the conjugate on a porous solid support.

Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present subject matter. The support material may have any structural configuration so long as the coupled molecule is capable of binding to a binding agent (e.g., an antibody). Thus, the support configuration may be spherical, as in a bead, or cylindrical, as in the inside surface of a test tube, or the external surface of a rod. Alternatively, the surface may be flat such as a plate (or a well within a multiwell plate), sheet, or test strip, etc. Preferred supports include polystyrene beads. Those skilled in the art will know many other suitable carriers for binding antibody or antigen, or will be able to ascertain the same by use of routine experimentation.

In embodiments, the agonists or antagonists may include small molecules. A small molecule is a compound that is less than 2000 Daltons in mass. The molecular mass of the small molecule is preferably less than 1000 Daltons, more preferably less than 600 Daltons, e.g., the compound is less than 500 Daltons, less than 400 Daltons, less than 300 Daltons, less than 200 Daltons, or less than 100 Daltons. Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecules can be synthetically engineered to consist of a fragment, or small portion, or a longer amino acid chain to fill a binding pocket of an enzyme. Typically, small molecules are less than one kiloDalton.

Described herein are anti-CTHRC1 antibodies. For example, monoclonal antibodies 10G07 (Duarte et al., 2014 PLOS ONE, 9(6): e100449, incorporated herein by reference), 13D11, and 19C07 are specific for the N terminus of human CTHRC1 and do not react with rat or murine CTHRC1. Anti-CTHRC1 antibody, clone 13E09, recognizes an epitope located within the N terminal half of the molecule of both human and rodent CTHRC1. Also provided is anti-CTHRC1 antibody, H-213, incorporated herein by reference and anti-CTHRC1 antibody. T-19, which is incorporated herein by reference (Santa Cruz Biotechnology, Inc., Dallas, Tex.). Also included are the following anti-CTHRC1 antibodies: SAB1102667, HPA059806, SAB2107469, and SAB1402656, each of which is incorporated herein by reference (Sigma-Aldrich®, St. Louis, Mo.). Also included is the following anti-CTHRC1 antibody PA5-38054, incorporated herein by reference (Thermo Scientific, Waltham, Mass.). In some cases, the anti-CTHRC1 antibodies described herein are administered at a concentration of 0.1 μg/ml to 500 mg/ml.

Also provided are anti-CTHRC1 antibodies, i.e., anti-GPR180 antibodies. For example, provided herein are the following anti-GPR180 antibodies: HPA047250, SAB4500617, SAB1303667, SAB1408931, each of which is incorporated herein by reference (Sigma-Aldrich®, St. Louis, Mo.). Also described herein is the following anti-GRP180 antibody: PA5-26788, incorporated herein by reference (Thermo Scientific, Waltham, Mass.). Also provided is an anti-GPR180 antibody, NBP2-14068, incorporated herein by reference (Novus Biologicals, Littleton, Colo.). Described herein is an anti-GPR180 antibody, ABIN952608, incorporated herein by reference (antibodies-online.com; Atlanta, Ga.). In some cases, the anti-GPR180 antibodies described herein are administered at a concentration of 0.1 μg/ml to 500 mg/ml.

Antibodies and fragments thereof described herein include, but are not limited to, polyclonal, monoclonal, chimeric, dAb (domain antibody), single chain, Fab, Fab′ and F(ab′)2 fragments, Fv, scFvs. A fragment of an antibody possess the immunological activity of its respective antibody. In some embodiments, a fragment of an antibody contains 1500 or less, 1250 of less, 1000 or less, 900 or less, 800 or less, 700 or less, 600 or less, 500 or less, 400 or less, 300 or less, 200 or less amino acids. For example, a protein or peptide inhibitor contains 1500 or less, 1250 of less, 1000 or less, 900 or less, 800 or less, 700 or less, 600 or less, 500 or less, 400 or less, 300 or less, 200 or less, 100 or less, 80 or less, 70 or less, 60 or less, 50 or less, 40 or less, 30 or less, 25 or less, 20 or less, 10 or less amino acids. For example, a nucleic acid inhibitor of the invention contains 400 or less, 300 or less, 200 or less, 150 or less, 100 or less, 90 or less, 80 or less, 70 or less, 60 or less, 50 or less, 40 or less, 35 or less, 30 or less, 28 or less, 26 or less, 24 or less, 22 or less, 20 or less, 18 or less, 16 or less, 14 or less, 12 or less, 10 or less nucleotides.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (κ) and lambda (λ), based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ) and mu (μ), respectively. The γ and α classes are further divided into subclasses on the basis of relatively minor differences in C_{H} sequence and function, e.g., humans express the following subclasses: IgG1, IgG2, IgG3, IgG4, IgA1, and IgA2. The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Also provided are variable domain antigen-binding sequences derived from human antibodies. Accordingly, chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc). Chimeric antibodies also include primatized and humanized antibodies.

Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

A “humanized antibody” is generally considered to be a human antibody that has one or more amino acid residues introduced into it from a source that is non-human. These non-human amino acid residues are often referred to as “import” residues, which are typically taken from an “import” variable domain. Humanization is traditionally performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting import hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance.

An “intact” antibody is one that comprises an antigen-binding site as well as a C_{L} and at least heavy chain constant domains, C_{H} 1, C_{H} 2 and C_{H} 3. The constant domains may be native sequence constant domains (e.g., human native sequence constant domains) or amino acid sequence variant thereof. Preferably, the intact antibody has one or more effector functions.

An “antibody fragment” comprises a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870; Zapata et al., Protein Eng. 8(10): 1057-1062 [1995]); single-chain antibody molecules; and multispecific antibodies formed from antibody fragments.

The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc_{ε}RI.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H} 1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

The “Fc” fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Detect” refers to identifying the presence, absence, or amount of the agent (e.g., a nucleic acid molecule, for example deoxyribonucleic acid (DNA) or ribonucleic acid (RNA), or mRNA or the protein) to be detected.

By “detectable label” is meant a composition that when linked (e.g., joined—directly or indirectly) to a molecule of interest renders the latter detectable, via, for example, spectroscopic, photochemical, biochemical, immunochemical, or chemical means. Direct labeling can occur through bonds or interactions that link the label to the molecule, and indirect labeling can occur through the use of a linker or bridging moiety which is either directly or indirectly labeled. Bridging moieties may amplify a detectable signal. For example, useful labels may include radioactive isotopes, magnetic beads, metallic beads, colloidal particles, fluorescent labeling compounds, electron-dense reagents, enzymes (for example, as commonly used in an enzyme-linked immunosorbent assay (ELISA)), biotin, digoxigenin, or haptens. When the fluorescently labeled molecule is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, p-phthaldehyde and fluorescamine. The molecule can also be detectably labeled using fluorescence emitting metals such as 152 Eu, or others of the lanthanide series. These metals can be attached to the molecule using such metal chelating groups as diethylenetriaminepentacetic acid (DTPA) or ethylenediaminetetraacetic acid (EDTA). The molecule also can be detectably labeled by coupling it to a chemiluminescent compound. The presence of the chemiluminescent-tagged molecule is then determined by detecting the presence of luminescence that arises during the course of chemical reaction. Examples of particularly useful chemiluminescent labeling compounds are luminol, isoluminol, theromatic acridinium ester, imidazole, acridinium salt and oxalate ester.

In some embodiments, detection is accomplished using an enzyme-linked immunosorbent assay (ELISA) or Western blot format. In other examples, the binding agent comprises a CTHRC1 specific nucleic acid (e.g., primers or a probe complementary for CTHRC1 RNA or cDNA), and the detecting step is accomplished using a polymerase chain reaction (PCR) or Northern blot format, or other means of detection. In various embodiments, a probe or primer is about 10-20, 15-25, 15-35, 15-25, 20-80, 50-100, or 10-100 nucleotides in length, e.g., about 10, 12, 15, 20, 25, 30, 35, 40, 45, 50, 55, 60, 70, 80, 90, or 100 nucleotides in length or less than about 30, 35, 40, 45, 50, 55, 60, 70, 80, 90, or 100 nucleotides in length.

A “detection step” may use any of a variety of known methods to detect the presence of nucleic acid. The types of detection methods in which probes can be used include Western blots, Southern blots, dot or slot blots, and Northern blots.

The term “immobilized” or “attached” refers to a probe (e.g., nucleic acid or protein) and a solid support in which the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule to the support and the non-covalent binding of a biotinylated probe to the molecule. Immobilization may also involve a combination of covalent and non-covalent interactions.

Biomarker changes that may be detected also include the redistribution of biomarkers (e.g., CTHRC1) between different compartments of a body, such as between a vessel and blood or between cerebrospinal fluid (CSF) and blood. Therefore, assaying of one or more of the biomarkers provided herein may be performed using a specific binding agent that may be detected with via imaging.

In various embodiments, a test (e.g., assay) is carried out on a bodily fluid such as blood, serum, plasma, saliva, tears, vitreous, cerebrospinal fluid, sweat, cerebrospinal fluid, or urine. The level of a protein may be measured using any applicable method known in the art, such as an immunoassay such as an enzyme-linked immunosorbent assay (ELISA), Western blot, radioimmunoassay (RIA), fluoroimmunoassay, or mass spectrometry. Non-limiting examples of mass spectrometry techniques include electrospray ionization (ESI), matrix assisted laser desorption (MALDI), MALDI-TOF (Time of flight), Fourier transform ion cyclotron resonance (FTIC), and surface-enhanced laser desorption (SELDI). Non-limiting examples of live imaging techniques for detecting the level and/or location (e.g., expression or localization changes) of a biomarker in the body of a subject include ultrasound, CT scans, an X-ray, MRI, PET, and SPECT.

As used herein, an antibody that “internalizes” is one that is taken up by (i.e., enters) the cell upon binding to an antigen on a mammalian cell (e.g., a cell surface polypeptide or receptor). The internalizing antibody will of course include antibody fragments, human or chimeric antibody, and antibody conjugates. For certain therapeutic applications, internalization in vivo is contemplated. The number of antibody molecules internalized will be sufficient or adequate to kill a cell or inhibit its growth, especially an infected cell. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the infected cell.

As used herein, an antibody is said to be “immunospecific,” “specific for” or to “specifically bind” an antigen if it reacts at a detectable level with the antigen, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and in certain embodiments, HuM2e antibody specifically binds to M2e if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Affinities of antibodies can be readily determined using conventional techniques, for example, those described by Scatchard et al. (Ann. N.Y. Acad. Sci. USA 51:660 (1949)).

Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

An antibody having a “biological characteristic” of a designated antibody is one that possesses one or more of the biological characteristics of that antibody which distinguish it from other antibodies. For example, in certain embodiments, an antibody with a biological characteristic of a designated antibody will bind the same epitope as that bound by the designated antibody and/or have a common effector function as the designated antibody.

The term “antagonist antibody” is used in the broadest sense, and includes an antibody that partially or fully blocks, inhibits, or neutralizes a biological activity of an epitope, polypeptide, or cell that it specifically binds. Methods for identifying antagonist antibodies may comprise contacting a polypeptide or cell specifically bound by a candidate antagonist antibody with the candidate antagonist antibody and measuring a detectable change in one or more biological activities normally associated with the polypeptide or cell.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

By “agent” is meant any small molecule chemical compound, antibody, nucleic acid molecule, or polypeptide, or fragments thereof.

By “Collagen Triple Helix Repeat Containing 1” or “Cthrc1” is meant a polypeptide having at least about 85%, e.g., at least about 90%, at least about 95%, or at least about 99%, sequence identity to NCBI Accession No. NP_AAQ89273, e.g., AAQ89273.1, or a fragment thereof that regulates metabolism. An exemplary sequence of human CTHRC1 is (SEQ ID NO: 1):

| | |
|---|---|
| 1 | mrpqgpaasp qrlrglllll llqlpapssa seipkgkqka qlrqrevvdl yngmclqgpa |
| 61 | gvpgrdgspg anvipgtpgi pgrdgfkgek geclresfee swtpnykqcs wsslnygidl |
| 121 | gkiaectftk mrsnsalrvl fsgslrlkcr naccqrwyft fngaecsgpl pieaiiyldq |
| 181 | gspemnstin ihrtssvegl cegigaglvd vaiwvgtcsd ypkgdastgw nsvsriiiee |
| 241 | lpk |

By a “nucleic acid encoding CTHRC1” is meant a nucleic acid having at least about 85%, e.g., at least about 90%, at least about 95%, or at least about 99%, sequence identity to NCBI Accession No. NM_138455 or NM_001256099. An exemplary nucleic acid encoding Cthrc1 is (SEQ ID NO: 2):

| | |
|---|---|
| 1 | gggagggaga gaggcgcgcg ggtgaaaggc gcattgatgc agcctgcggc ggcctcggag |
| 61 | cgcggcggag ccagacgctg accacgttcc tctcctcggt ctcctccgcc tccagctccg |
| 121 | cgctgcccgg cagccgggag ccatgcgacc ccagggcccc gccgcctccc cgcagcggct |
| 181 | ccgcggcctc ctgctgctcc tgctgctgca gctgcccgcg ccgtcgagcg cctctgagat |
| 241 | ccccaagggg aagcaaaagg cgcagctccg gcagagggag gtggtggacc tgtataatgg |
| 301 | aatgtgctta caagggccag caggagtgcc tggtcgagac gggagccctg gggccaatgg |
| 361 | cattccgggt acacctggga tcccaggtcg ggatggattc aaaggagaaa agggggaatg |
| 421 | tctgagggaa agctttgagg agtcctggac acccaactac aagcagtgtt catggagttc |
| 481 | attgaattat ggcatagatc ttgggaaaat tgcggagtgt acatttacaa agatgcgttc |
| 541 | aaatagtgct ctaagagttt tgttcagtgg ctcacttcgg ctaaaatgca gaaatgcatg |
| 601 | ctgtcagcgt tggtatttca cattcaatgg agctgaatgt tcaggacctc ttcccattga |
| 661 | agctataatt tatttggacc aaggaagccc tgaaatgaat tcaacaatta atattcatcg |
| 721 | cacttcttct gtggaaggac tttgtgaagg aattggtgct ggattagtgg atgttgctat |
| 781 | ctgggttggt acttgttcag attacccaaa aggagatgct tctactggat ggaattcagt |
| 841 | ttctcgcatc attattgaag aactaccaaa ataaatgctt taattttcat ttgctacctc |
| 901 | tttttttatt atgccttgga atggttcact taaatgacat tttaaataag tttatgtata |
| 961 | catctgaatg aaaagcaaag ctaaatatgt ttacagacca aagtgtgatt tcacactgtt |
| 1021 | tttaaatcta gcattattca ttttgcttca atcaaaagtg gtttcaatat tttttttagt |
| 1081 | tggttagaat actttcttca tagtcacatt ctctcaacct ataatttgga atattgttgt |
| 1141 | ggtcttttgt tttttctctt agtatagcat ttttaaaaaa atataaaagc taccaatctt |
| 1201 | tgtacaattt gtaaatgtta agaatttttt ttatatctgt taaataaaaa ttatttccaa |
| 1261 | caaccttaat atctttaaa |

Another exemplary nucleic acid encoding CTHRC1 is (SEQ ID NO: 3):

| | |
|---|---|
| 1 | agaaggttta aggccggaaa gggaaatgaa ggggcccggc gctaaccctc taaggacctg |
| 61 | ttttgcttct gtttaaacca aatgggcagt ctgtcattac acacaccctg ggtcttcata |
| 121 | tgtggccgcc aggtaggagc atcacagtca agctacggga gaaaacagtt tccaggaaac |
| 181 | tggaaatgaa cggcccgagt gctttccagg ggctcatctg tgggaagtat aatggaatgt |
| 241 | gcttacaagg gccagcagga gtgcctggtc gagacgggag ccctggggcc aatggcattc |
| 301 | cgggtacacc tgggatccca ggtcgggatg gattcaaagg agaaaagggg gaatgtctga |
| 361 | gggaaagctt tgaggagtcc tggacaccca actacaagca gtgttcatgg agttcattga |
| 421 | attatggcat agatcttggg aaaattgcgg agtgtacatt tacaaagatg cgttcaaata |
| 481 | gtgctctaag agttttgttc agtggctcac ttcggctaaa atgcagaaat gcatgctgtc |
| 541 | agcgttggta tttcacattc aatggagctg aatgttcagg acctcttccc attgaagcta |
| 601 | taatttattt ggaccaagga agccctgaaa tgaattcaac aattaatatt catcgcactt |
| 661 | cttctgtgga aggactttgt gaaggaattg gtgctggatt agtggatgtt gctatctggg |
| 721 | ttggtacttg ttcagattac ccaaaaggag atgcttctac tggatggaat tcagtttctc |
| 781 | gcatcattat tgaagaacta ccaaaataaa tgctttaatt ttcatttgct acctcttttt |
| 841 | ttattatgcc ttggaatggt tcacttaaat gacattttaa ataagtttat gtatacatct |
| 901 | gaatgaaaag caaagctaaa tatgtttaca gaccaaagtg tgatttcaca ctgtttttaa |
| 961 | atctagcatt attcattttg cttcaatcaa aagtggtttc aatatttttt ttagttggtt |
| 1021 | agaatacttt cttcatagtc acattctctc aacctataat ttggaatatt gttgtggtct |
| 1081 | tttgtttttt ctcttagtat agcattttta aaaaaatata aaagctacca atctttgtac |
| 1141 | aatttgtaaa tgttaagaat tttttttata tctgttaaat aaaaattatt tccaacaacc |
| 1201 | ttaatatctt taaa |

By “control” or “reference” is meant a standard of comparison. As used herein, “changed as compared to a control” sample or subject is understood as having a level of the analyte or diagnostic or therapeutic indicator to be detected at a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, (3-galactosidase or luciferase). Depending on the method used for detection, the amount and measurement of the change can vary. Determination of statistical significance is within the ability of those skilled in the art, e.g., the number of standard deviations from the mean that constitute a positive result.

Relative to a control level, the level that is determined may be an increased level. As used herein, the term “increased” with respect to level (e.g., expression level, biological activity level, e.g., mRNA level of CTHRC1 or protein level of CTHRC1, etc.) refers to any % increase above a control level. The increased level may be at least or about a 1% increase, at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level.

As used herein, “detecting” and “detection” are understood that an assay performed for identification of a specific analyte in a sample, e.g., an antigen in a sample or the level of an antigen in a sample. The amount of analyte or activity detected in the sample can be none or below the level of detection of the assay or method.

By “diagnosing” as used herein refers to a clinical or other assessment of the condition of a subject based on observation, testing, or circumstances for identifying a subject having a disease, disorder, or condition based on the presence of at least one indicator, such as a sign or symptom of the disease, disorder, or condition. Typically, diagnosing using the method of the invention includes the observation of the subject for multiple indicators of the disease, disorder, or condition in conjunction with the methods provided herein. A diagnostic method provides an indicator that a disease is or is not present. A single diagnostic test typically does not provide a definitive conclusion regarding the disease state of the subject being tested.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “polynucleotide” or “nucleic acid” as used herein designates mRNA, RNA, cRNA, cDNA or DNA. As used herein, a “nucleic acid encoding a polypeptide” is understood as any possible nucleic acid that upon (transcription and) translation would result in a polypeptide of the desired sequence. The degeneracy of the nucleic acid code is well understood. Further, it is well known that various organisms have preferred codon usage, etc. Determination of a nucleic acid sequence to encode any polypeptide is well within the ability of those of skill in the art.

In some cases, a compound (e.g., small molecule) or macromolecule (e.g., nucleic acid, polypeptide, or protein) of the invention is purified and/or isolated. As used herein, an “isolated” or “purified” small molecule, nucleic acid molecule, polynucleotide, polypeptide, or protein (e.g., antibody or fragment thereof), is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA), e.g., synthetic cDNA) is free of the genes or sequences that flank it in its naturally occurring state. Purified also defines a degree of sterility that is safe for administration to a human subject, e.g., lacking infectious or toxic agents.

Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. Similarly, an isolated nucleic acid is removed from its normal physiological environment. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. An isolated virus or viral vector is a virus that is removed from the cells, typically in culture, in which the virus was produced.

By “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (α) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

As used herein, “kits” are understood to contain at least one non-standard laboratory reagent for use in the methods of the invention in appropriate packaging, optionally containing instructions for use. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100.mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In an embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Obtaining” is understood herein as manufacturing, purchasing, or otherwise coming into possession of.

As used herein, “operably linked” is understood as joined, preferably by a covalent linkage, e.g., joining an amino-terminus of one peptide, e.g., expressing an enzyme, to a carboxy terminus of another peptide, e.g., expressing a signal sequence to target the protein to a specific cellular compartment; joining a promoter sequence with a protein coding sequence, in a manner that the two or more components that are operably linked either retain their original activity, or gain an activity upon joining such that the activity of the operably linked portions can be assayed and have detectable activity, e.g., enzymatic activity, protein expression activity.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, a-tocopherol, and the like; and metal chelating agents, such as citric acid, ethylenediamine tetraacetic acid (EDTA), sorbitol, tartaric acid, phosphoric acid, and the like. Formulations of the present invention include those suitable for oral, nasal, topical, transdermal, buccal, sublingual, intramuscular, intracardiac, intraperotineal, intrathecal, intracranial, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, “plurality” is understood to mean more than one. For example, a plurality refers to at least two, three, four, five, or more.

A “polypeptide” or “peptide” as used herein is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full length proteins (e.g., fully processed proteins) as well as shorter amino acids sequences (e.g., fragments of naturally occurring proteins or synthetic polypeptide fragments). Optionally the peptide further includes one or more modifications such as modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins, Structure and Molecular Properties, 2nd ed., T. E. Creighton, W.H. Freeman and Company, New York (1993); Posttranslational Covalent Modification of Proteins, B. C. Johnson, ed., Academic Press, New York, pgs. 1-12 (1983); Seifter et al., Meth. Enzymol 182:626-646 (1990); Rattan et al., Ann. N.Y. Acad. Sci. 663:48-62 (1992)).

The term “reduce” or “increase” is meant to alter negatively or positively, respectively, by at least 5%. An alteration may be by 5%, 10%, 25%, 30%, 50%, 75%, or even by 100%.

A “sample” as used herein refers to a biological material that is isolated from its environment (e.g., blood or tissue from an animal, cells, or conditioned media from tissue culture) and is suspected of containing, or known to contain an analyte, such as a protein. A sample can also be a partially purified fraction of a tissue or bodily fluid. A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

A “solid support” describes a strip, a polymer, a bead, or a nanoparticle

A “subject” as used herein refers to an organism. In certain embodiments, the organism is an animal. In certain embodiments, the subject is a living organism. In certain embodiments, the subject is a cadaver organism. In certain preferred embodiments, the subject is a mammal, including, but not limited to, a human or non-human mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A “subject sample” can be a sample obtained from any subject, typically a blood or serum sample, however the method contemplates the use of any body fluid or tissue from a subject. The sample may be obtained, for example, for diagnosis of a specific individual for the presence or absence of a particular disease or condition.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with diminished cardiac function is within the ability of those in the art. Subjects suffering from, and suspected of suffering from, a specific disease, condition, or syndrome are not necessarily two distinct groups.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range. This includes all individual sequences when a range of SEQ ID NOs: is provided. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Unless specifically stated or obvious from context, as used herein, the term “or” is understood to be inclusive. Unless specifically stated or obvious from context, as used herein, the terms “a”, “an”, and “the” are understood to be singular or plural.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is a schematic showing the development of Cthrc1 null and Cthrc1 transgenic mouse models to assess the in vivo role of CTHRC1 in adipogenesis and regulation of body composition.

FIG. 2A and FIG. 2B are a series of dot plots that illustrate that high CTHRC1 correlates with low body fat in humans. FIG. 2A is a dot plot wherein a collaboration with Drs. Elaine Lee (UConn) and Frank Suhr (Deutsche Sporthochschule Köln) found high CTHRC1 in ironman athletes and cyclists participating in the HHH100 (Hotter'N Hell100), and even higher levels in ultra-marathon runners (2009 Trans-Europe Foot Race, 4500 km in 64 days¹³), compared to healthy volunteers. FIG. 2B is a dot plot showing that CTHRC1 levels in Ironman athletes before the race were similar to levels <1 h after the race and the levels revealed a significant inverse correlation to amount of body fat in that individual. Subjects were male, 21±3 years of age.

FIG. 3A, FIG. 3B, and FIG. 3C are a series of photomicrographs and a dot plot showing that CTHRC1 levels inversely correlate to body fat in mice. FIG. 3A-FIG. 3B show MicroCT scans of a wildtype (FIG. 3A) or Cthrc1 null (FIG. 3B) mouse showing excess visceral fat with loss of CTHRC1³. FIG. 3C is a dot plot showing circulating CTHRC1 in transgenic mice in relation to body fat. Data were obtained in male mice and similar studies using female mice are ongoing.

FIG. 4A, FIG. 4B, and FIG. 4C are a series of photomicrographs and a dot plot showing transgenic expression of CTHRC1 in liver elevates circulating plasma levels. Tg(CAG-GFP-hCTHRC1)^{Vli} transgenics on the Cthrc1 null background were crossed with Alb^{tm1}(cre/ERT2)^{Mtz} mice. FIG. 4A is a photomicrograph that shows, by immunostaining, CTHRC1 was only in double positive mice given tamoxifen (TMX). FIG. 4B is a photomicrographs howing that no CTHRC1 was detected in Cre negative mice after TMX injection. FIG. 4C is a dot plot wherein circulating transgenic CTHRC1 was quantified using the human specific ELISA. Significant elevation of CTHRC1 plasma levels was achieved. Data shown in FIG. 4A-FIG. 4B were from female mice and data in FIG. 4C are from male and female mice. As described in the approach, transgenic studies continue to use both male and female mice. Additional control groups include mice with the same genotype that received vehicle (corn oil) instead of tamoxifen.

FIG. 5A-FIG. 5D is a series of photomicrographs showing histology of visceral WAT (FIG. 5A and FIG. 5B) and interscapular BAT (FIG. 5C and FIG. 5D) from a Cthrc1 transgenic mouse with 20 ng/ml circulating CTHRC1 levels (FIG. 5A and FIG. 5C) and a control transgenic mouse with CTHRC1 levels below detection (FIG. 5B and FIG. 5D). WAT in FIG. 5A contains increased numbers of small adipocytes and increased stromal cells compared to FIG. 5B. Lipid droplets of BAT in FIG. 5C, however, appear larger, less multilocular, and more beige-like than in the control transgenic mouse FIG. 5D. Mice were 2 male littermates and tamoxifen was injected after weaning to induce the transgene.

FIG. 6A-FIG. 6G is a series of graphs showing CTHRC1 suppresses adipogenic signaling in white preadipocytes. Immortalized Cthrc1 null preadipocytes transduced with LacZ or Cthrc1 were induced to differentiate, and PPARγ (FIG. 6A) and SREBP1c (FIG. 6B) levels determined by immunoblotting in non-differentiated cells or 48 h (PPARγ) or 168 h (SREBP1c) after differentiation. 3T3-L1 cells transduced with LacZ or Cthrc1 were induced to differentiate, and C/EBPδ (FIG. 6C) and C/EBPα (FIG. 6D) mRNA quantified by qRT-PCR in non-differentiated cells and 48 h after differentiation. FIG. 6E shows immortalized brown adipocytes transduced with LacZ or Cthrc1 were induced to differentiate, and stained with Oil red 0 after 96 h. PPARγ (FIG. 6F) in non-differentiated cells and 48 h after the onset of brown adipogenic differentiation was determined by immunoblotting. FIG. 6G shows 3T3-L1 cells transduced with LacZ or Cthrc1 were induced to differentiate and CHOP levels were determined by immunoblotting at the indicated time points. Representative data of 3 separate experiments are shown in each figure.

FIG. 7A-FIG. 7C is a series of photomicrographs and a photograph of an immunoblot showing evidence for interaction between GPR180 and CTHRC1. CHO-K1 cells that do not express significant levels of endogenous Gpr180 were transfected with empty vector (FIG. 7A) or Gpr180-myc (FIG. 7B) and treated with CTHRC1 protein for 10 min on ice followed by immunostaining to detect CTHRC1 binding. The cells with expression of GPR180 selectively bound CTHRC1 protein, which was detected by immunostaining (red). FIG. 7C shows RT-PCR results showing that Gpr180 is widely expressed in tissues and cell lines including 3T3-L1 cells and white adipose tissue (WAT).

FIG. 8 is a photograph of an immunoblot showing targeting of Gpr180. PCR detection of LoxP integration sites in 11 pups after CRISPR/Cas9 targeting of Gpr180. A LoxP specific forward primer and Gpr180 specific primers for the upper (top panel) and lower (bottom panel) integration sites were used.

FIG. 9A-FIG. 9B is a series of photomicrographs showing that pituitary CTHRC1 correlates to parental nutrition. In FIG. 9A, cells in the anterior pituitary from a one month old rat exposed to protein malnutrition conditions in utero. In FIG. 9B, a pituitary gland from a control rat derived from a pregnancy under normal dietary conditions (25% protein) shows no CTHRC1. Rats were raised by mothers on a 25% protein diet.

FIG. 10 is a dot plot showing testosterone levels in females with detectable versus non-detectable CTHRC1 levels.

### DETAILED DESCRIPTION

The invention is based, at least in part, on the surprising discovery that CTHRC1 levels predict endurance athletic performance. As described herein, the highest levels of circulating CTHRC1 are observed in champion endurance athletes. As described in detail below, healthy human subjects can have high circulating levels of CTHRC1 without engaging in endurance athletic activity. Furthermore, subjects who engage regularly in endurance athletic exercise can have levels of CTHRC1 that are below the detection limit. As such, as described in detail below, CTHRC1 levels may therefore be genetically or epigenetically determined. In healthy woman age 20-35 with a body mass index of ≤25, CTHRC1 levels had a positive correlation with testosterone levels. Higher testosterone levels can make a big difference in exercise performance in woman. The effect of exercise on CTHRC1 levels is examined. Described herein is the evaluation of CTHRC1 levels in ultramarathon runners, bicycles racers of HHH100, and ironman athletes.

Specifically, described herein is the identification of the effects of CTHRC1 on body fat, voluntary physical activity, and energy expenditure. Unique mouse gain- and loss-of-function models are utilized. Also described is the identification of the signaling mechanism by which CTHRC1 regulates adipogenesis. CTHRC1 signaling is characterized through GPR180 and downstream targets are identified. Finally, described herein is the assessment of regulation of CTHRC1 via a nutritional, trans-generational effect. It is also determined whether this regulation affects body composition in offspring.

In recent years, the focus of health research has shifted to developing and testing behavioral, surgical, and pharmaceutical interventions for the obesity epidemic¹. Yet, substantial opportunities remain in clarifying the molecular and cellular mechanisms related to adipogenesis, control of body mass, and regulation of physical activity and metabolism. Harnessing these mechanisms may be key to effectively addressing the growing financial and healthcare burden of this epidemic. Major causes of obesity include societal and environmental factors, including a sedentary lifestyle. In addition, genetics alter susceptibility to obesity. Yet, few effective interventions exist for preventing or treating obesity cost-effectively. In the primary care setting, sustained behavioral modification is difficult to achieve, hard to sustain, and has not proven effective. Similarly, existing surgical and pharmacotherapy approaches have risks that make them narrowly appropriate. There is a tremendous need to identify and evaluate therapeutic targets for the treatment and prevention of obesity.

Described herein is the identification of Cthrc1 (collagen triple helix repeat containing-1) and its role as a metabolic regulator²⁻⁴. Also, translational studies were performed to correlate CTHRC1 to metabolic changes related to human physiology. The results presented herein indicate that this is a high priority candidate for anti-obesity strategies. As described in detail below, the mechanisms by which CTHRC1 controls adipogenesis and metabolism are identified. Thus, the results presented herein use unique mouse pre-clinical models, which are used for both physiology and cell signaling experiments. The global Cthrc1 null mouse strain develops increased body fat with reduced muscle mass without changes in total body weight, similar to changes associated with aging in humans. In addition, Cthrc1 null mice exhibit a 50% reduction in voluntary physical activity and reduced muscle strength compared to wildtype mice. Because preadipocytes express and respond to CTHRC1, adipose tissue is a primary target of CTHRC1 signaling. Indeed, CTHRC1 inhibited white adipocyte differentiation along with expression of key pro-adipogenic transcription factors while preadipocytes isolated from Cthrc1 null mice show enhanced white adipogenic differentiation. The results presented herein suggest that endogenous CTHRC1 suppresses adipogenesis and promotes a lean phenotype. Described herein is the mechanism of this regulation.

To understand the signaling mechanism of CTHRC1, a candidate CTHRC1 receptor, the G-protein coupled receptor GPR180, which is broadly expressed, including in adipose tissue was identified. Thus, described herein is the identification of downstream signaling events to confirm GPR180 as a functional CTHRC1 receptor. Since GPR180 was an orphan receptor, this is a major advance in understanding contributions of both CTHRC1 and GPR180 in regulation of overall metabolism and adipogenesis. Described herein is the investigation of two aspects of CTHRC1 biology: 1) its in vivo regulation of body fat and energy expenditure and 2) its signaling mechanism underlying the regulation of adipogenesis.

Obesity and Metabolic Disease are Major Health Issues with No Effective Treatments.

Obesity is a body mass index of ≥30, and the prevalence of obesity is 34.95% of adults in the USA¹. Minority subgroups are disproportionately affected, with over 56% of African Americans and over 44% of Hispanic Americans being obese. Obesity causes a huge medical and financial burden, and is associated with increased incidence of type 2 diabetes, metabolic syndrome, and cardiovascular diseases. Major causes of obesity are societal and environmental factors, including a sedentary lifestyle⁵ and high food availability (ers.usda.gov/data). In addition, genetics alter susceptibility to obesity⁶. Unfortunately, there are few effective interventions to prevent or treat obesity. One such intervention, sustained behavioral modification, is often recommended but is difficult to achieve⁷. Indeed, behavioral treatment for obesity in the primary care setting has not proven to be effective⁸, while surgical⁹ and pharmacotherapy approaches^{10, 11} have associated risk factors. Thus, clarifying molecular and cellular mechanisms related to adipogenesis, control of body mass, and regulation of physical activity and metabolism is key to effectively addressing the growing financial and healthcare burden of this epidemic.

### Collagen Triple Helix Repeat Containing-1 (Cthrc1) Protein

Collagen triple helix repeat containing 1 (Cthrc1) was originally discovered in a screen for sequences induced in injured arteries, where it was identified that Cthrc1 expressed in adventitial cells of remodeling arteries but not in uninjured vessels. Subsequent studies also demonstrated that CTHRC1 is characteristically expressed by the activated fibroblast associated with wound healing as well as cancer-activated fibroblast. Kimura et al. first reported that Cthrc1 null mice exhibit reduced bone mass, and in vitro osteogenic differentiation of bone marrow stromal cells revealed that endogenously expressed Cthrc1 is required for effective osteogenic differentiation by affecting cell proliferation and differentiation. In contrast, it was recently reported that CTHRC1 stimulates bone formation in vitro and that it functions as a coupling factor in vivo, produced by mature actively resorbing osteoclasts. The key difference between the two studies is the identity of the CTHRC1-producing cell, which in the absence of specific and reliable antibody reagents has remained controversial.

Collagen triple helix repeat containing-1 (CTHRC1) is a protein isolated from a cDNA library of injured arteries. CTHRC1 functions as an inhibitor of TGF-β signaling. CTHRC1 is susceptible to cleavage by proteases and purified CTHRC1 forms aggregates, making it difficult to perform cell binding studies and protein interaction studies. Expression analyses of CTHRC1 in tissues have been performed by in situ hybridization, immunohistochemistry and RT-PCR analysis. CTHRC1 has also been found in plasma. CTHRC1 plasma levels in healthy human volunteers ranged from 16-440 ng/ml.

An exemplary sequence of human CTHRC1 is (SEQ ID NO: 1):

| | |
|---|---|
| 1 | mrpqgpaasp qrlrglllll llqlpapssa seipkgkqka qlrqrevvdl yngmclqgpa |
| 61 | gvpgrdgspg anvipgtpgi pgrdgfkgek geclresfee swtpnykqcs wsslnygidl |
| 121 | gkiaectftk mrsnsalrvl fsgslrlkcr naccqrwyft fngaecsgpl pieaiiyldq |
| 181 | gspemnstin ihrtssvegl cegigaglvd vaiwvgtcsd ypkgdastgw nsvsriiiee |
| 241 | lpk |

An exemplary nucleic acid encoding Cthrc1 is (SEQ ID NO: 2):

| | |
|---|---|
| 1 | gggagggaga gaggcgcgcg ggtgaaaggc gcattgatgc agcctgcggc ggcctcggag |
| 61 | cgcggcggag ccagacgctg accacgttcc tctcctcggt ctcctccgcc tccagctccg |
| 121 | cgctgcccgg cagccgggag ccatgcgacc ccagggcccc gccgcctccc cgcagcggct |
| 181 | ccgcggcctc ctgctgctcc tgctgctgca gctgcccgcg ccgtcgagcg cctctgagat |
| 241 | ccccaagggg aagcaaaagg cgcagctccg gcagagggag gtggtggacc tgtataatgg |
| 301 | aatgtgctta caagggccag caggagtgcc tggtcgagac gggagccctg gggccaatgg |
| 361 | cattccgggt acacctggga tcccaggtcg ggatggattc aaaggagaaa agggggaatg |
| 421 | tctgagggaa agctttgagg agtcctggac acccaactac aagcagtgtt catggagttc |
| 481 | attgaattat ggcatagatc ttgggaaaat tgcggagtgt acatttacaa agatgcgttc |
| 541 | aaatagtgct ctaagagttt tgttcagtgg ctcacttcgg ctaaaatgca gaaatgcatg |
| 601 | ctgtcagcgt tggtatttca cattcaatgg agctgaatgt tcaggacctc ttcccattga |
| 661 | agctataatt tatttggacc aaggaagccc tgaaatgaat tcaacaatta atattcatcg |
| 721 | cacttcttct gtggaaggac tttgtgaagg aattggtgct ggattagtgg atgttgctat |
| 781 | ctgggttggt acttgttcag attacccaaa aggagatgct tctactggat ggaattcagt |
| 841 | ttctcgcatc attattgaag aactaccaaa ataaatgctt taattttcat ttgctacctc |
| 901 | tttttttatt atgccttgga atggttcact taaatgacat tttaaataag tttatgtata |
| 961 | catctgaatg aaaagcaaag ctaaatatgt ttacagacca aagtgtgatt tcacactgtt |
| 1021 | tttaaatcta gcattattca ttttgcttca atcaaaagtg gtttcaatat tttttttagt |
| 1081 | tggttagaat actttcttca tagtcacatt ctctcaacct ataatttgga atattgttgt |
| 1141 | ggtcttttgt tttttctctt agtatagcat ttttaaaaaa atataaaagc taccaatctt |
| 1201 | tgtacaattt gtaaatgtta agaatttttt ttatatctgt taaataaaaa ttatttccaa |
| 1261 | caaccttaat atctttaaa |

Another exemplary nucleic acid encoding CTHRC1 is (SEQ ID NO: 3):

| | |
|---|---|
| 1 | agaaggttta aggccggaaa gggaaatgaa ggggcccggc gctaaccctc taaggacctg |
| 61 | ttttgcttct gtttaaacca aatgggcagt ctgtcattac acacaccctg ggtcttcata |
| 121 | tgtggccgcc aggtaggagc atcacagtca agctacggga gaaaacagtt tccaggaaac |
| 181 | tggaaatgaa cggcccgagt gctttccagg ggctcatctg tgggaagtat aatggaatgt |
| 241 | gcttacaagg gccagcagga gtgcctggtc gagacgggag ccctggggcc aatggcattc |
| 301 | cgggtacacc tgggatccca ggtcgggatg gattcaaagg agaaaagggg gaatgtctga |
| 361 | gggaaagctt tgaggagtcc tggacaccca actacaagca gtgttcatgg agttcattga |
| 421 | attatggcat agatcttggg aaaattgcgg agtgtacatt tacaaagatg cgttcaaata |
| 481 | gtgctctaag agttttgttc agtggctcac ttcggctaaa atgcagaaat gcatgctgtc |
| 541 | agcgttggta tttcacattc aatggagctg aatgttcagg acctcttccc attgaagcta |
| 601 | taatttattt ggaccaagga agccctgaaa tgaattcaac aattaatatt catcgcactt |
| 661 | cttctgtgga aggactttgt gaaggaattg gtgctggatt agtggatgtt gctatctggg |
| 721 | ttggtacttg ttcagattac ccaaaaggag atgcttctac tggatggaat tcagtttctc |
| 781 | gcatcattat tgaagaacta ccaaaataaa tgctttaatt ttcatttgct acctcttttt |
| 841 | ttattatgcc ttggaatggt tcacttaaat gacattttaa ataagtttat gtatacatct |
| 901 | gaatgaaaag caaagctaaa tatgtttaca gaccaaagtg tgatttcaca ctgtttttaa |
| 961 | atctagcatt attcattttg cttcaatcaa aagtggtttc aatatttttt ttagttggtt |
| 1021 | agaatacttt cttcatagtc acattctctc aacctataat ttggaatatt gttgtggtct |
| 1081 | tttgtttttt ctcttagtat agcattttta aaaaaatata aaagctacca atctttgtac |
| 1141 | aatttgtaaa tgttaagaat tttttttata tctgttaaat aaaaattatt tccaacaacc |
| 1201 | ttaatatctt taaa |

### CTHRC1 as an Anti-Obesity Modulator of Metabolism and Adipogenesis

Described herein is a hormonal regulator of adiposity and metabolism. Described herein is the development and characterization of gain- and loss-of-function mouse strains and the determination of translational relevance by analysis of human populations. Also described herein is the development and characterization of specific reagents to study CTHRC1 function, including specific monoclonal antibodies and sensitive ELISAs to quantify human and mouse CTHRC1. In humans, substantial levels of CTHRC1 are only found in a minority of individuals, and notably, endurance athletes with low adiposity are highly represented in this population. The pre-clinical, cellular and molecular studies show that CTHRC1 efficiently blocks adipogenesis, and it was identified as a candidate cellular receptor. Finally, the phenotypes of the CTHRC1 mouse strains are strong models, since they are consistent with the relationship of CTHRC1 and adiposity in humans.

Described herein is the identification of CTHRC1 and its hormonal functions as an endogenous inhibitor of adipocyte differentiation. Its candidate receptor is GPR180. CTHRC1 regulates body composition, adipocyte physiology, and voluntary physical activity. These effects are highly relevant to the roots of obesity as a public health problem. In this study, the physiologic role of this factor in adipogenesis is characterized and its role in physiology is evaluated. The results provided herein have broad implications for obesity, physical activity, muscle physiology, muscle loss and adiposity related to aging, and obesity-related cardiovascular diseases.

Additionally, described herein is the generation of highly specific CTHRC1 monoclonal antibodies for various applications. The sandwich ELISAs described herein are the only ones currently able to measure CTHRC1 plasma levels in humans and rodents. These assays are critical for the evaluation of CTHRC1 as a biomarker. Screening plasma samples from thousands of human subjects revealed CTHRC1 levels below detection (<30 pg/ml) for most subjects, while ˜20% had much higher levels (up to 100 ng/ml)¹². The examples provided below generate entirely new information on the physiology of CTHRC1 in mice and humans. As described herein, CTHRC1 suppresses adipogenic differentiation of white, but not brown, preadipocytes, and immortalized cultures of Cthrc1 null white preadipocytes have been developed that will be used to determine the molecular mechanisms of CTHRC1's anti-adipogenic effect. As described in detail below, in preadipocytes stimulated to differentiate CTHRC1 downregulates the expression of pro-adipogenic transcription factors C/EBPδ, C/EBPα and PPARγ, while enhancing the expression of the anti-adipogenic regulator CHOP.

CTHRC1 is the first ligand identified for the orphan receptor GPR180. As described herein, characterization of this interaction is essential for development of small molecule agonists or antagonists. Agonists may hold potential in pharmacological approaches for the treatment of obesity and low voluntary physical activity. Notably, G protein-coupled receptors are among the targets most suited for drug development. Described herein is the development of Cre-inducible transgenic mouse strains on wildtype and Cthrc1 null backgrounds. Using tamoxifen-inducible albumin-Cre mice, levels of circulating CTHRC1 observed in humans are mimicked. In addition, using suitable Cre mice Cthrc1 is selectively re-expressed in specific tissues to examine the differential effects of circulating versus locally expressed CTHRC1 in normal physiology.

As described in detail below, using CRISPR/Cas9, a foxed Gpr180 allele and a global Gpr180 null mouse was generated, thereby demonstrating the use of cutting edge technologies to advance the science. Within the institutional mass spectrometry core facility, state-of-the-art, unbiased lipidomics using ion scanning and fragmentation mass spectrometry of all precursor species (MS/MS^{ALL}) are performed.

### Pharmaceutical Compositions and Administration

The present invention comprises pharmaceutical preparations comprising a human CTHRC1 agonist (or antagonist) polypeptide together with a pharmaceutically acceptable carrier. Such compositions are useful for the treatment or prevention of fatty liver disease, low bone mass, and muscle weakness, or for the prevention or treatment of any one or more of the risk factors associated with these conditions. Polypeptides of the invention may be administered as part of a pharmaceutical composition. The compositions should be sterile and contain a therapeutically effective amount of the polypeptides in a unit of weight or volume suitable for administration to a subject.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups also can be derived from inorganic bases such as, for example, sodium, potassium, ammonium, calcium or ferric hydroxides, and such organic bases as isopropylamine, trimethyl amine, 2-ethylamino ethanol, histidine, procaine and the like. Particularly preferred are the salts of TFA and HCl.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions also can contain liquid phases in addition to and to the exclusion of water. Exemplary of such additional liquid phases are glycerin, vegetable oils such as cottonseed oil, and water-oil emulsions.

A therapeutic composition contains an inflammation inhibiting amount or a fibrosis inhibiting amount of an Cthrc1 polypeptide of the present invention, typically formulated to contain an amount of at least 0.1 weight percent of Cthrc1 polypeptide per weight of total therapeutic composition. A weight percent is a ratio by weight of inhibitor to total composition. Thus, for example, 0.1 weight percent is 0.1 grams of inhibitor per 100 grams of total composition.

These compositions can be stored in unit or multi-dose containers, for example, sealed ampoules or vials, as an aqueous solution or as a lyophilized formulation for reconstitution. As an example of a lyophilized formulation, 10 mL vials are filled with 5 mL of sterile-filtered 1% (w/v) aqueous Cthrc1 polypeptide solution, and the resulting mixture can then be lyophilized. The infusion solution can be prepared by reconstituting the lyophilized material using sterile Water-for-Injection (WFI).

The compositions can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

The dosage ranges for the administration of the Cthrc1 polypeptide vary. In general, amounts are large enough to produce the desired effect in which disease symptoms of a metabolic syndrome are ameliorated. The dosage should not be so large as to cause adverse side effects. Generally, the dosage will vary with the age, condition, sex and extent of the disease in the patient and can be determined by one of skill in the art. The dosage also can be adjusted by the individual physician in the event of any complication.

A therapeutically effective amount is an amount sufficient to produce a measurable inhibition of symptoms of a condition (e.g., an increase in bone mass or a decrease in muscle weakness). Such symptoms are measured in conjunction with assessment of related clinical parameters.

A therapeutically effective amount of a polypeptide of this invention in the form of a polypeptide, or fragment thereof, is typically an amount of polypeptide such that when administered in a physiologically tolerable composition is sufficient to achieve a plasma concentration of from about 0.1 microgram (μg) per milliliter (mL) to about 200 μg/mL, or from about 1 ug/mL to about 150 ug/mL. In one embodiment, the plasma concentration in molarity is from about 2 micromolar (uM) to about 5 millimolar (mM) or from 100 uM to 1 mM Cthrc1 polypeptide. In other embodiments, the doses of polypeptide ranges from about 500 mg/Kg to about 1.0 g/kg (e.g., 500, 600, 700, 750, 800, 900, 1000 mg/kg).

The agents of the invention can be administered parenterally by injection or by gradual infusion over time. In other embodiments, agents are administered intravenously, intraperitoneally, intramuscularly, subcutaneously, intracavity, transdermally, topically, intraocularly, orally, intranasally, and can be delivered by peristaltic means. In one embodiment, a therapeutic compositions containing an agent of this invention are administered in a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered and timing depends on the patient to be treated, capacity of the patient's system to utilize the active ingredient, and degree of therapeutic effect desired. Precise amounts of active ingredient required to be administered depend on the judgement of the practitioner and are peculiar to each individual. However, suitable dosage ranges for systemic application are disclosed herein and depend on the route of administration. Suitable regimes for administration also are variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

The amount and frequency of administration of antibody would depend on a number of factors including, but not limited to, the condition to be treated.

### Kits

The invention provides kits for the treatment or prevention of a metabolic syndrome. In one embodiment, the kit includes a therapeutic or prophylactic composition containing an effective amount of an agent described herein. In some embodiments, the kit comprises a sterile container that contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

### Rigor and Consideration of Sex and Other Biological Variables

The results presented herein adhere to high quality experimentation, unbiased analysis and interpretation, and transparent dissemination, and follow NIH-recommended guidelines^{18,19}. General principles include use of documented protocols and validated procedures, appropriate controls, technical and biological replication, and blinded data collection. Described herein is the utilization of female and male mice in equivalent proportions, as recommended in NIH guidelines^{20,21}. The source, as well as age, gender, weight, environmental housing conditions, underlying health conditions, and any treatments will be carefully documented and confirmed. To increase reproducibility, rigorous experimental design is used to a priori calculate sample size, identify inclusion/exclusion criteria, establish methods for randomization, and determine how to handle missing data. It is not known whether there will be significant sex differences in all of the phenotypes. Thus, data is analyzed blinded to experimental group with genders separated (male vs females) and as pooled samples. To properly power these studies, it is assumed that there will be sex differences, so that required sample size for statistical significance is fulfilled in both gender groups. In preliminary studies, genders were analyzed separately because of this. Using the two group Satterthwaite t-test of equal means, using the least dramatic difference for a conservative estimate, it was calculated that a sample size of 8 in each group (unless stated otherwise) will have 80% power (p<0.05) to detect a significant difference in means of the parameters to be studied. The mice are studied as young (8 weeks of age) and older adults (16 weeks of age) to reflect adult physiology, and pilot studies showed that the adipocyte and metabolic phenotypes are well established at these ages. In preliminary studies in humans, neither gender nor age showed an association with circulating plasma CTHRC1 levels¹², but a gender difference is queried for in mouse models. For cell studies, the adipocyte progenitor cells derived from the Cthrc1 null mice are thoroughly characterized. These cells spontaneously immortalized in culture after several weeks. These cells have been characterized for gene expression, quantitative and qualitative content of lipids (mass-spectrometry) and ability to differentiate into adipocytes. They have the same features as primary preadipocytes. For statistical analysis of data, ANOVA is used to compare multiple groups to determine a statistical significance at p<0.05, using GraphPad Prism software. Lipid analyses is performed using LipidView, PeakView and MarkerView.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1: Development of Cthrc1 Null and Cthrc1 Transgenic Mouse Models to Assess the In Vivo Role of CTHRC1 in Adipogenesis and Regulation of Body Composition

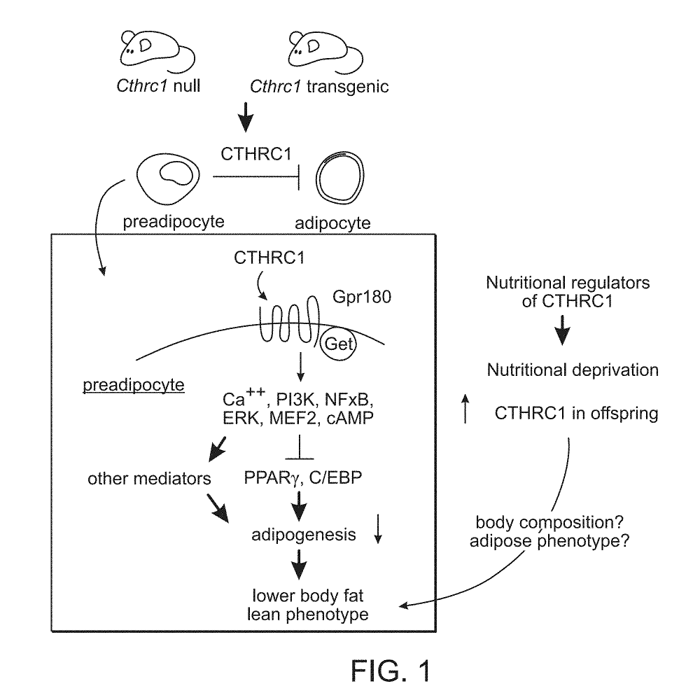

Described herein is the signaling mechanism underlying the regulation of adipogenesis (FIG. 1). These models show cellular and metabolic differences consistent with an endogenous role for CTHRC1 as an anti-obesity hormone. As described herein, one primary target is the adipocyte lineage, and normal CTHRC1 activity suppresses adipogenesis. As described in detail below, the mouse models characterize body composition, metabolic activity, and adipogenesis, and the impact of CTHRC1. Also described below are the signaling mechanisms underlying the regulation of adipogenesis, focusing on the recent discovery of an interaction between CTHRC1 and GPR180. Finally, it was identified that nutritional status of a pregnant female affects the production of CTHRC1 in offspring. As described in detail below, it is examined whether this represents a trans-generational control of body composition, despite normal diet and genotype of the offspring.

### Example 2: CTHRC1 is Elevated and Associated with the Level of Body Fat in Elite Endurance Athletes

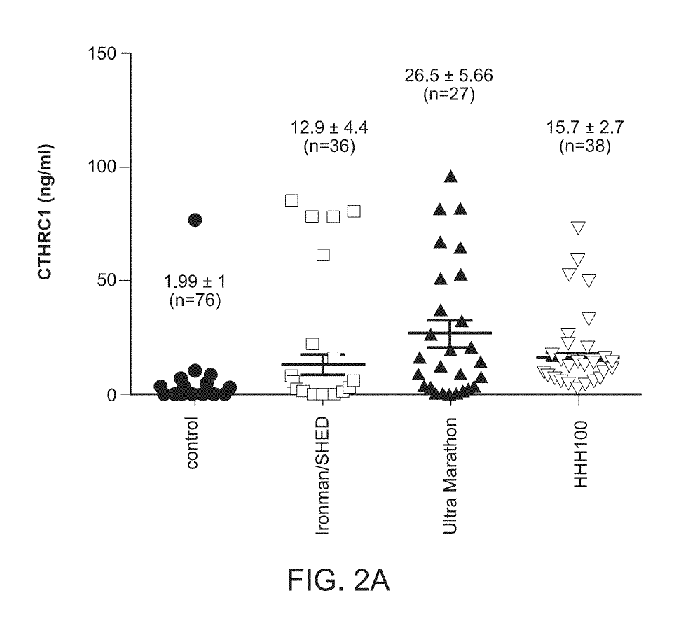

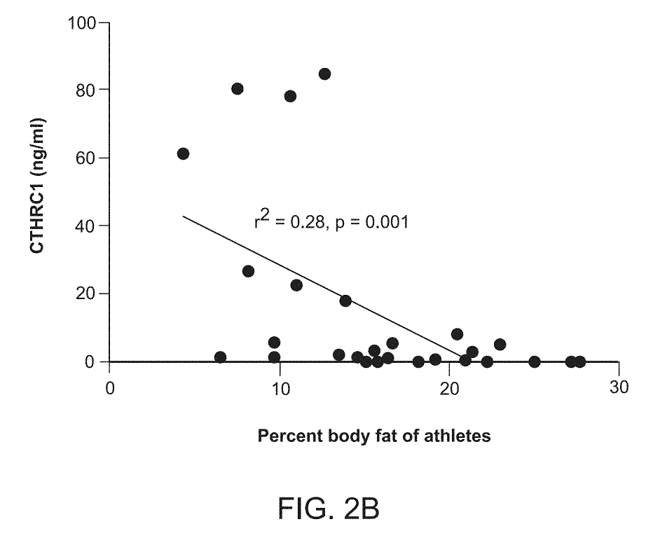

It was recently identified that CTHRC1⁴, is a circulating hormone that regulates metabolism². A sensitive ELISA was developed to quantify human CTHRC1¹², which detects only non-degraded CTHRC1. In most healthy humans, CTHRC1 circulates at concentrations below the lower detection limit of the assay. However, some healthy adults have much higher levels, which ultimately led us to discover an additional link between athletic status and CTHRC1. The majority of competitive endurance athletes tested have extremely high CTHRC1 levels (FIG. 2A) and there is a significant inverse correlation of CTHRC1 levels with body fat in endurance athletes (FIG. 2B).

Endurance athletes have distinct metabolic characteristics and tend to have low adiposity, which allowed for the detection of these significant changes in circulating CTHRC1 levels. In humans, lower BMI and lower body fat are associated with faster Ironman race times^{14, 15}. A study of ultramarathon participants (161 km) showed that faster men have lower percent body fat values than slower competitors, and the values ranged from 6 to 14% for the fastest 3 runners¹⁶. Translational studies provide a strong indication that metabolic status and body composition, specifically of adipose tissue, may be associated with the levels of circulating CTHRC1. This exciting finding has potential to open new avenues of discovery related to whether CTHRC1 could be a hormonal treatment to mediate anti-adiposity effects in obese individuals or those with metabolic disease. As described herein, to provide more evidence that endogenous CTHRC1 may indeed be a target for anti-obesity therapies, pre-clinical mouse models were developed and characterized.

### Example 3: Metabolic and Adipose Tissue Phenotypes in Genetic Mouse Models

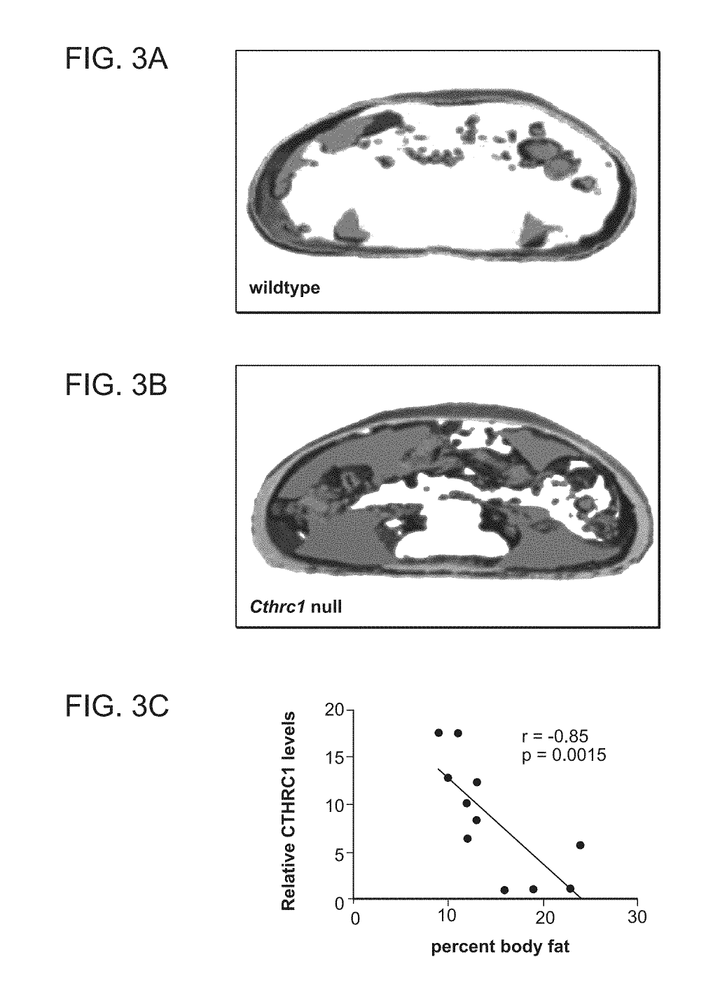

To understand the causal roles of CTHRC1 in metabolism, a mouse model was developed with a null deletion of the Cthrc1 gene². The Cthrc1 null mice developed fatty livers², suggesting a problem with lipid handling or metabolism. Whole body composition was analyzed using NMR (Bruker Minispec) and microCT, and comprehensive metabolic and activity monitoring (Promethion, Sable Systems) was performed in a Physiology Core Facility³. Differences between wildtype and Cthrc1 null mice are summarized in Table 1. Cthrc1 null mice have similar body weight but increased body fat (FIG. 3A-FIG. 3B) and decreased lean mass (muscle), showing altered body composition. Similar to results in humans (FIG. 2B), there is a strong inverse correlation in mice between circulating CTHRC1 levels and body fat (FIG. 3C). Thus, the Cthrc1 null mice metabolic phenotype is consistent with observations in humans.

TABLE 1
Loss of CTHRC1 in mice results in metabolic changes,
altered body composition and reduced voluntary physical activity.

| | Wildtype Mice | Cthrc1 Null Mice | p value |
|---|---|---|---|
| Body Weight (g) | 28.80 ± 0.51 | 28.80 ± 1.03 | 1.0 |
| Body Fat (g) | 3.92 ± 0.21 | 7.57 ± 0.74 | 0.0003 |
| Lean Body Mass (g) | 22.91 ± 0.49 | 19.81 ± 0.48 | 0.0007 |
| Food Consumption (g/24 h) | 3.56 ± 0.18 | 2.55 ± 0.18 | 0.0019 |
| Running Wheel Speed (average, | 0.263 ± 0.014 | 0.201 ± 0.015 | 0.033 |
| m/s) | | | |
| Running Distance (m/24 h) | 6164 ± 370 | 3346 ± 607 | 0.0013 |
| 24 h Avg. Energy Expenditure | 0.588 ± 0.010 | 0.524 ± 0.022 | 0.0154 |
| (EE) | (kcal/h) | (kcal/h) | |
| 24 h Average RQ | 0.860 ± 0.010 | 0.828 ± 0.0097 | 0.043 |
| Still per 24 h (%) | 56.28 ± 1.66 | 65.82 ± 1.86 | 0.0021 |
| Grip Strength (N/g) | 0.03542 ± 0.001305 | 0.02806 ± 0.001868 | 0.002 |

In Table 1, metabolic and activity monitoring was performed on 11 week old wildtype and Cthrc1 null male mice (n=8/group). Grip strength (n=38 for wildtype and n=19 for Cthrc1 null mice) was determined in 16 week old mice³. Muscle fiber analyses showed no abnormalities and no CTHRC1 expression in muscle³. Ongoing parallel studies using female mice are in progress and data indicate that results are similar for females.

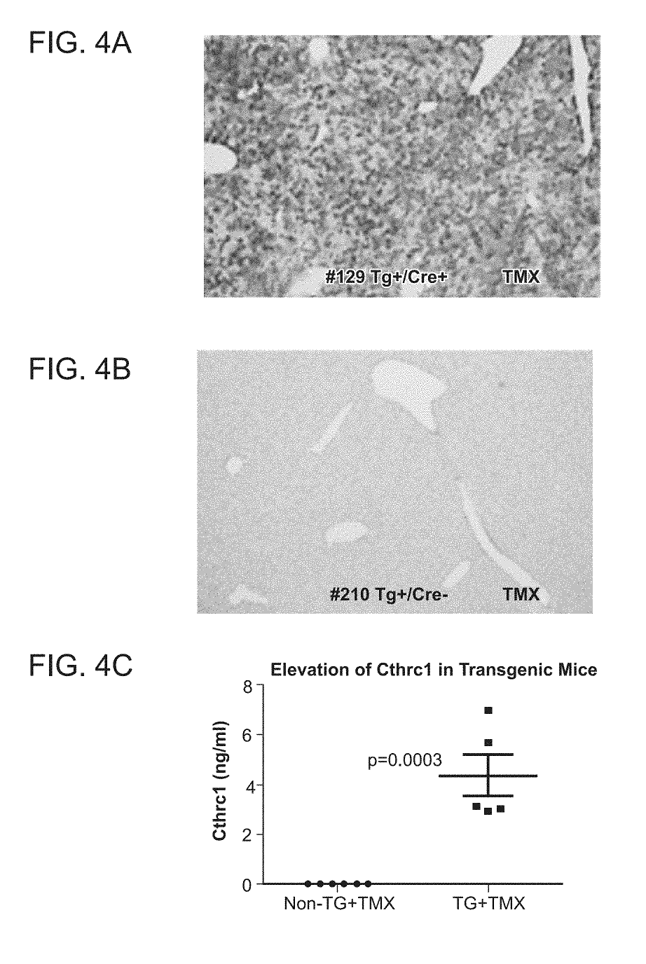

In addition to the Cthrc1 null deletion, a Cthrc1 transgenic mouse line was characterized with conditional expression of human CTHRC1 (Tg(CagGfp-hCTHRC1)^{Vli 12}. In this mouse, human CTHRC1 follows a foxed GFP-stop sequence that is constitutively active under a CMV promoter/actin enhancer. Breeding to a tissue-specific Cre driver allows for conditional expression of the CTHRC1 transgene. Mice with conditional vascular expression of the transgene had reduced body fat (FIG. 3C)³. To carefully regulate CTHRC1 levels, the conditional transgenic line was crossed onto the Cthrc1 null background. Expression was evaluated using a liver-specific Cre recombinase strain (albumin promoter, FIG. 4A-FIG. 4C, Alb^{tm1}(cre/ERT2)^{Mtz 17}). This transgene allows for mimicing elevated CTHRC1 plasma levels observed in ˜20% of human subjects (based on >2000 samples analyzed). Both the loss- and gain-of-function models will be utilized to clarify in vivo effects of circulating CTHRC1.

### Example 4: To Determine the Effects of CTHRC1 on Body Fat, Voluntary Physical Activity, and Energy Expenditure

Unique mouse gain- and loss-of-function models are utilized herein. CTHRC1 is expressed constitutively in bone and brain², and is localized to neuroendocrine cells of the hypothalamus and posterior pituitary in response to nutritional status, and is circulating in the blood. Its hormonal activity affects adipose tissue, skeletal muscle, liver, and bone, all tissues involved in metabolic regulation. CTHRC1 has peripheral effects mediated by circulating CTHRC1, in addition to potential local effects in tissues where it is produced, such as the bone, brain, and adipose tissue. Preliminary studies in mouse models have shown intriguing changes in adiposity and metabolism regulated by CTHRC1.

### Circulating CTHRC1 has Anti-Adipogenic Activity and Beneficial Metabolic Effects.

Significant effort was invested in developing and characterizing mouse models to advance knowledge of the in vivo roles of CTHRC1. The Tg(CAG-GFP-hCTHRC1)^{Vli} strain on the Cthrc1 null background, using Albtm1(cre/ERT2)^{Mtz 17} mice is utilized to increase circulating levels of hCTHRC1. The optimal tamoxifen dose for this experiment is determined by monitoring hCTHRC1 plasma levels, and corn oil is used as a vehicle control in double transgenic mice. Body composition and size of white fat depots (inguinal, gonadal, retroperitoneal) and brown interscapular adipose (control) is determined at 8 weeks of age before induction of the transgene, and 8 weeks after induction of the transgene (16 weeks of age), separately for male and female mice (n=8/group, see power calculation above). Further analysis of gene expression, adipose tissue histology, and physiology is performed using the experimental design in Table 2. Analysis is performed 8 weeks after transgene induction.

TABLE 2
Overview of experimental design

| | | Expected | |
|---|---|---|---|
| Mouse Strain | Treatment | Condition | Analysis |
| Tg(CAG-GFP- | tamoxifen | transgenic | NMR - body composition (fat and lean mass) |
| hCTHRC1)^{VI};AlbCre; | | hCTHRC1 | microCT - in vivo, adipose tissue volume of visceral |
| Cthrc1 null | | induced | and subcutaneous depots³ |
| Tg(CAG-GFP- | corn oil | control (will | body weight |
| hCTHRC1)^{VI};AlbCre; | control | detect leakiness | quantify circulating CTHRC1 (ELISA) |
| Cthrc1 null | | of inducible | adipose weight and histology (adipocyte size, number) |
| | | transgene) | oil red O staining of adipose depots |
| | | | adipose gene expression (qRT-PCR and immunoblot |
| | | | for PPARγ and its downstream targets including aP2, |
| | | | and UCP1 to test for beiging²²) |
| | | | adipose tissue triglyceride content using mass |
| | | | spectrometry²³ |
| Tg(CAG-GFP- | tamoxifen | no CTHRC1 (no | initial studies of phenotype by NMR to determine if |
| hCTHRC1)^{VI}; Cthrc1 | | Cre), tamoxifen | there are changes in body composition. |
| null | | control | |
| Tg(CAG-GFP- | corn oil | no CTHRC1 (no | If it appears that tamoxifen has a significant effect on |
| hCTHRC1)^{VI}; | control | Cre), tamoxifen | body fat or adipocyte phenotype, these controls will be |
| Cthrc1 null | | control | critical to understand the specific effects of CTHRC1, |
| | | | and the complete phenotyping panel will be performed |
| | | | for comparison and statistical analysis with the |
| | | | CTHRC1 transgenic strains. |

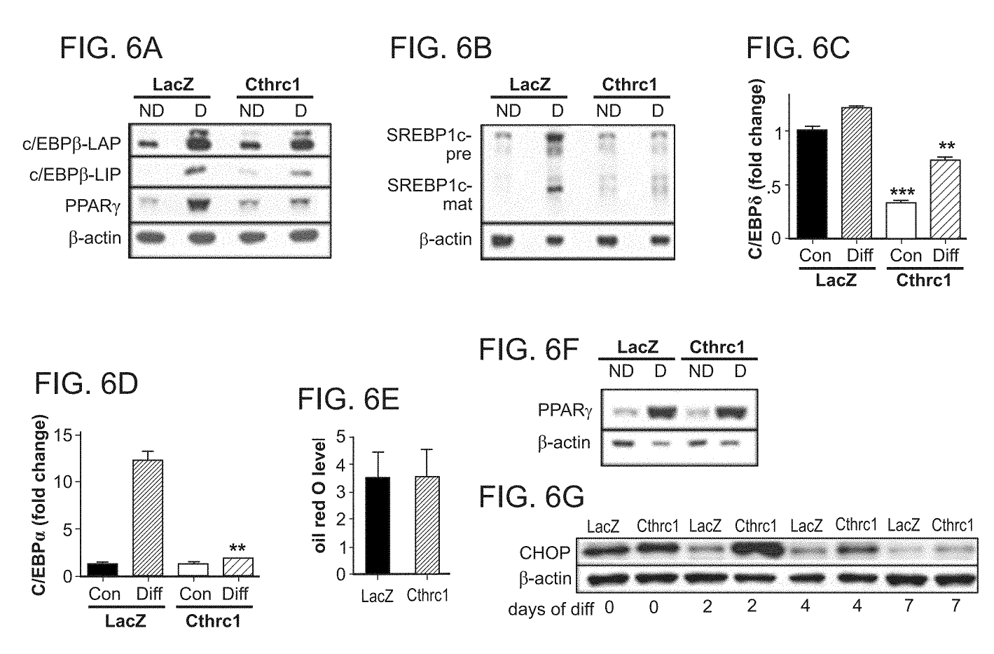

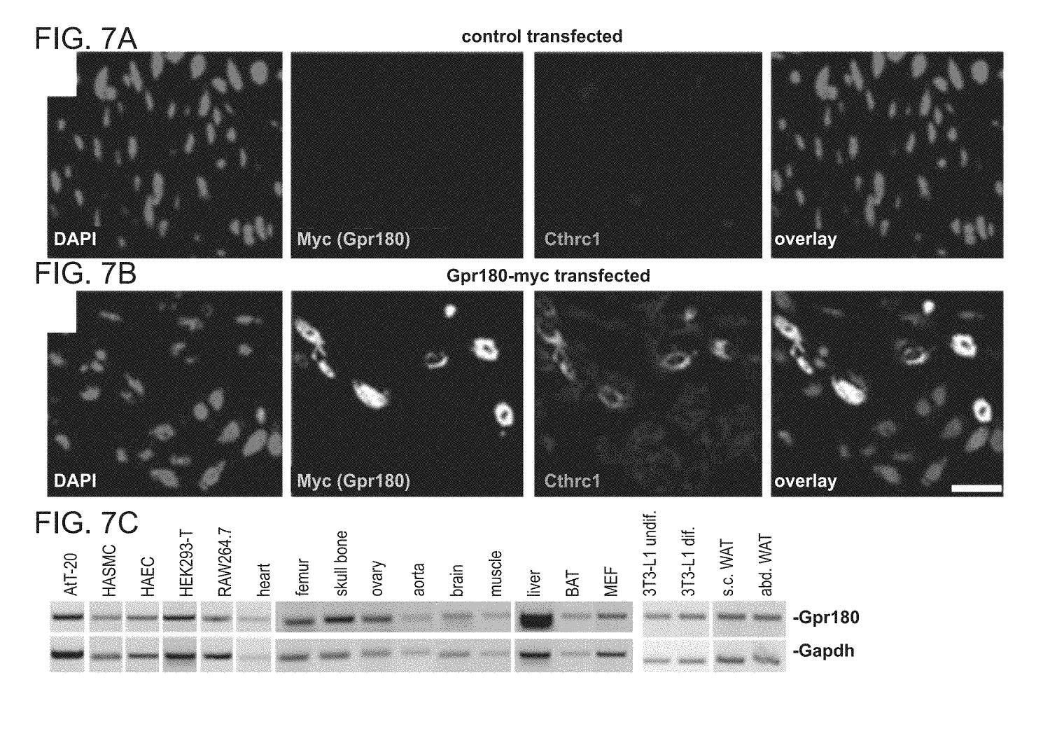

It is not expected that increased CTHRC1 levels will result in increased Ucp1 expression, because this would increase energy expenditure (EE), as a matter of fact the opposite may occur. This expectation is also supported by the absence of CTHRC1 effects on brown adipogenesis in vitro (FIG. 6E-FIG. 6F). Adipocyte number may be determined during adolescence before the age of 8 weeks, and thus not change due to elevation of CTHRC1 in the adult. This is addressed by increasing CTHRC1 levels later during development or during early postnatal development (E18.5-P14). The preliminary data showing inhibition of adipocyte differentiation suggest that CTHRC1 may be influencing adipocyte number before adulthood. If fat cell number differs among groups, determine adipocyte turnover, i.e. adipocyte death (TUNEL, activated caspase-3 staining), macrophage infiltration and surrounding of adipocytes (“crowning,” F4/80 staining). In addition, adipocyte proliferation is determined by chronic BrdU administration (in drinking water for 2 weeks) and BrdU immunohistochemistry following the labeling period. It is anticipated that raising plasma levels of the CTHRC1 early during postnatal development may result in reduced adipocyte number and smaller fat depots. Downregulated Ppary mRNA and protein may be observed with an inverse relationship to circulating CTHRC1. Although CTHRC1 plasma levels in mice may not be as high as those in some athletes, the increases achieved raise the levels several orders of magnitude above those of wildtype mice, which is sufficient for functional analysis. Also, by overexpressing the transgene on the null background, potentially confounding local functions that endogenous CTHRC1 may exhibit in the brain are eliminated.

### Metabolic and Activity Monitoring

Using the Physiology Core Facility, indirect calorimetry and activity monitoring³ in CTHRC1 transgenic mice before and after induction of the transgene (Table 2) is utlilized. Data on EE, respiratory quotient (RQ), distance in running wheel, and average wheel speed is determined as in Table 1. Ambulatory activity and wheel-running is determined simultaneously with collection of calorimetry data. Correlations are assessed between EE, lean mass, food consumption, and wheel activity to determine if these variables impact changes in energy expenditure²⁵⁻²⁷. Close attention is payed to EE at rest normalized to lean mass because this parameter will not be confounded by the differences in activity (wheel running, walking etc.). Preliminary data are expanded and in-depth metabolic and activity monitoring is performed by including additional Cthrc1 null, wildtype, and gain-of-function mice (FIG. 4A-FIG. 4C) in these analyses (n=8 per genotype and gender). With the inducible transgene on the Cthrc1 null background, metabolic and activity parameters are measured before induction of the transgene with tamoxifen (8 weeks of age) and after induction of the transgene (16 weeks) to determine the effects of elevated circulating CTHRC1. CTHRC1 transgenic mice on the Cthrc1 null background (but no Cre transgene) injected with tamoxifen will serve as controls (Table 2). Data collection over a 5-day period in the metabolic cages is performed and mice with similar circulating CTHRC1 levels will be compared. In addition to the genetic approaches, mice are intravenously twice daily with 1 μg of CTHRC1 and their resting EE is measured. The half-life of CTHRC is 1 2.5 h in circulation and based on that a twice-daily dose should be sufficient.

### CTHRC1 Effects on Adipose Tissue Composition

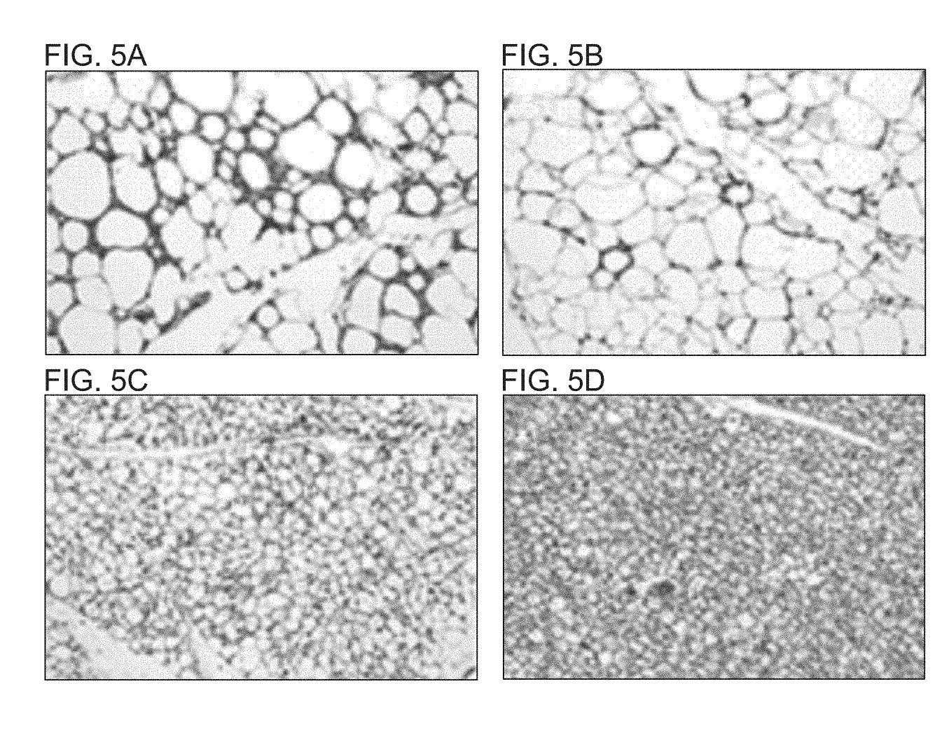

Determining the underlying mechanism for the observed phenotype in Cthrc1 null mice (Table 1) is a high priority. A preliminary analysis of white and brown adipose tissue (WAT, BAT) from transgenic mice with high levels of circulating CTHRC1 (FIG. 5A and FIG. 5B) revealed increased cellularity of WAT stroma and a decrease of white adipocyte size, whereas the BAT from the same mouse showed larger lipid droplets in adipocytes compared to typical BAT morphology seen in the control mice (FIG. 5C and FIG. 5D). Based on inhibition of white adipogenesis in vitro by CTHRC1 demonstrated earlier³, one possibility is that the accumulated WAT stromal cells in FIG. 5A represent various precursors prevented from differentiating into adipocytes by the elevated circulating CTHRC1 levels. These stromal cells are characterized and quantified by multicolor immunofluorescence confocal microscopy using specific combinations of markers to determine stromal vascular cells (Sca-1, Pdgfrb, CD36), adipose stem cells (Sca-1, FGF2, VEGF-D), committed preadipocytes (NG2, Pref-1, PPARγ)²⁸ and myeloid lineage-derived cells (F4/80, myeloperoxidase, CD11b). In parallel, RNA is isolated from WAT, and qRT-PCR of marker gene expression is used to compare the abundance of the above mentioned cell populations. Using co-immunostaining for the endothelial cell marker (VE-cadherin), the association of adipose stem cells and preadipocytes with blood vessels is determined. The adipocyte-free stromal-vascular fraction of WAT cells is produced, immunofluorescently stained with specific combinations of antibodies to cell surface markers²⁸ and the abundance of adipose stem cells and preadipocytes using flow cytometry (MMCRI core facility) is analyzed. Next, the stem cell potential of the stromal-vascular WAT cell population is determined by assessing their ability to differentiate into myocytes, osteoblasts or chondrocytes. If it turns out that higher levels of CTHRC1 are associated with increased numbers of adipogenic stem cells, this could be due to CTHRC1 effects on cell survival or proliferation. Therefore, in vivo EdU labeling studies (in drinking water up to 7 days) are performed as well as measurements of apoptosis (activated caspase-3) in WAT. Immunofluorescence confocal microscopy is used to co-localize the incorporated EdU or cleaved caspase 3 with the markers of adipose stem cells and preadipocytes. All adipose depots (inguinal, retroperitoneal, visceral) are analyzed in the manner described above (n=8/group, male and female). Preliminary quantification of PAX7 positive satellite cells in skeletal muscle, revealed that muscle from Cthrc1 null mice had an approx. 50% reduction in these cells (9.2±1.8 vs 16.3±2.0 cells per field, p=0.018), supporting a role for CTHRC1 in the positive regulation of the stem cell pool. These findings may have major implications for the stem cell and aging research field.

Differences in the morphology of the BAT of Cthrc1 transgenic mice (FIG. 5B and FIG. 5D), with more beige-like appearance in mice showing high CTHRC1 levels, prompted the examination of the effect of Cthrc1 on gene expression in BAT. It may be identified by qRT-PCR and immunostaining that there is less Ucp1 expression in BAT from mice with high CTHRC1 levels. Linking this back to the endurance athlete, it would conceptually make sense to have less Ucp1 in BAT because limited energy substrates could be redirected to fuel additional muscle work, which is thermogenic on its own. Finally, mass-spectroscopy is utilized to analyze the effects of CTHRC1 on the lipid spectra in WAT and BAT.

### Expected Results, Potential Problems, and Alternatives

Using the inducible transgenic approach and the most efficient liver-specific Cre driver line available (Albtm1(cre/ERT2)^{Mtz)}), the feasibility of elucidating the functions of circulating CTHRC1 in regulating body composition and activity was demonstrated. From levels below the detection limit in normal mice (<0.03 ng/ml), increases in plasma CTHRC1 up to 20 ng/ml can be achieved, which are physiologically relevant concentrations seen in humans. Intravenous injection of CTHRC1 expressing AAV also results in CTHRC1 expression in the liver and is an alternative for increasing circulating CTHRC1 as does driving the transgene with Tie2-Cre in endothelial cells (currently being tested). It is anticipated that adipose tissue depots will be smaller in Cthrc1 overexpressing mice and that adipocytes may be smaller in size or reduced in number or both. A reduction in number would suggest that CTHRC1 inhibits adipocyte differentiation from precursors, which may depend on the timing of the induction of the transgene. It might be determined that overexpression of CTHRC1 during embryonic development will have even greater effects on inhibition of adipose tissue formation. If voluntary physical activity does not change in mice with increased CTHRC1 levels (on Cthrc1 null background), it would suggest that CTHRC1 expressed in the brain regulates physical activity. Ongoing studies that may be given priority near the mid-point of this project include functionally characterizing a conditional Gpr180 allele with loss of function in the brain (see below). This targeting of the CTHRC1 receptor in the brain will clarify which processes are under the control of central CTHRC1. Central effects of CTHRC1 and whether circulating CTHRC1 crosses the blood-brain barrier will be determined in the future. For understanding the functions of the CTHRC1 hormone, the functions of circulating CTHRC1 in normal physiology were elucidated before introducing potentially confounding variables associated with disease.

The lack of a conditional null allele is not a concern because with conditional transgenic expression of CTHRC1 on the global null background, the roles of circulating CTHRC1 can be more precisely defined. Indeed, in this case, influences of CTHRC1 produced at sites of tissue remodeling or incomplete gene inactivation using a Cre/loxP approach is not a concern. Preliminary studies in humans found a positive correlation of CTHRC1 levels with peripheral dopamine production but not adrenaline or noradrenaline (24 h urine sample). Treatment with dopamine receptor agonists increases muscle mass and force²⁹ and decreases adiposity³⁰. Potential positive regulation of dopamine by CTHRC1 would be consistent with reduced muscle/lean mass and increased fat mass observed in Cthrc1 null mice. This is interesting because increased sympathetic tone via noradrenaline would not be able to explain the BAT phenotype in the transgenic mice. Future studies will address a potential CTHRC1/dopamine connection in more depth. The proof-of-principle studies described here are absolutely necessary for evaluation of CTHRC1 as a potential target for treating obesity and perhaps even age-related muscle wasting and frailty, as the reduction in satellite cells in Cthrc1 null mice might suggest.

### Example 5: The Signaling Mechanism by which CTHRC1 Regulates Adipogenesis

CTHRC1 effects on the adipogenic signaling cascade and verify CTHRC1 signaling through GPR180 are characterized. CTHRC1 is secreted and suppresses adipogenesis and expression of key adipogenic transcription factors (FIG. 6)³. It remains to be determined what upstream stage(s) of pro-adipogenic signaling are regulated by CTHRC1, and if CTHRC1 affects lipid biosynthesis pathways and mitochondrial activation associated with lipogenesis. The authors were unable to reproduce the effects of CTHRC1 on the Wnt and PCP pathway nor were they able to demonstrate CTHRC1 expression in the inner ear as reported by Yamamoto et al³¹. In addition, extensive testing of inner ear function in Cthrc1 null mice by collaborators revealed no abnormalities. Furthermore, most of the publications of CTHRC1 in cancer use insufficiently characterized CTHRC1 antibodies¹², raising concern about validity of many of the published studies. Over the course of 16 years, extensive knowledge about the properties of CTHRC1 was obtained, e.g., its susceptibility to proteolytic cleavage especially when studied in HEK293 cell-based assays as well as its tendency to form insoluble aggregates. Described herein are monoclonal antibodies that are rigorously validated using CTHRC1 positive transgenic specimens as well as CTHRC1 negative specimens from Cthrc1 null mice. Based on these considerations, the underlying mechanisms are elucidated in adipocyte-related cells, for which there exists both in vivo and in vitro data that they are responsive to CTHRC1.

Using the monoclonal antibodies, GPR180 was detected with >45% peptide sequence coverage in an affinity purification/mass-spectrometry approach of Cthrc1 null tissue lysates incubated with CTHRC1. GPR180 is an orphan GPCR, and has no known ligand or proven function. The data strongly suggest that GPR180 is a CTHRC1 receptor. The discovery of Cthrc1 (in rats)⁴ and Tsukuda's discovery of Gpr180³² were both in balloon-injured arteries. Gpr180 null mice reportedly develop normally, and are resistant to neointima formation in the femoral artery cuff model³², however, detailed analyses of bone, adipose tissue, physical activity and body composition in Gpr180 null mice have not been reported. Direct binding of CTHRC1 to GPR180 leading to receptor activation is verified herein. Furthermore, the role of GPR180 in CTHRC1-mediated effects on adipogenesis and identification the CTHRC1-induced signaling pathways is investigated.

As described herein, CTHRC1 inhibits specific stages of adipogenic differentiation, resulting in the suppression of lipid biosynthesis. CTHRC1 effects are mediated by the GPR180 receptor. To study CTHRC1 regulation of adipogenesis, spontaneously immortalized preadipocytes isolated from the inguinal adipose depot of Cthrc1 null mice are used. Three clones of Cthrc1 null immortalized preadipocytes have been produced, and they exhibit strong inducible adipogenic differentiation³, similar to primary Cthrc1 null preadipocytes. An efficient production system for CTHRC1 protein in conditioned medium from CHO cells adenovirally transduced with a Cthrc1 or β-gal (control) expression vector was developed. Typically, 3 μg/ml CTHRC1 is obtained in the conditioned medium, and a 1:10 dilution is used for experiments, maintaining a physiological concentration. This method is similar to that used for Wnt ligand generation in conditioned medium. Because CTHRC1 readily forms aggregates, especially at higher concentrations, it was determined that working with recombinant purified CTHRC1 is problematic. In parallel to experiments with conditioned medium, the direct adenoviral overexpression of CTHRC1 in preadipocytes is used.

### How is the Adipogenic Transcription Factor Cascade Regulated by CTHRC1?

Induction of adipogenesis requires a cascade of transcription factors³³. Shortly after induction of adipogenesis, transcription factor CREB is activated by phosphorylation, which enhances the transcription of C/EBPβ. Later, C/EBPβ acquires DNA binding activity as a result of specific phosphorylation and in cooperation with C/EBPδ stimulates the expression of PPARγ and C/EBPα. These transcription factors stimulate the expression of SREBP1, the key regulator of lipogenesis. It was demonstrated that CTHRC1 significantly inhibits the transcriptional activity of both CREB and PPARγ³. While qRT-PCR showed that CTHRC1 decreased C/EBPδ mRNA content both before and after the induction of differentiation (FIG. 6C), it did not affect the level of C/EBPβ (FIG. 6A). Strong inhibition of PPARγ and C/EBPα expression was detected in preadipocytes induced to differentiate (FIG. 6A and FIG. 6D). It was found that CTHRC1 strongly suppresses the levels of both immature and mature forms of SREBP1c, which is downstream of PPARγ and C/EBPα (FIG. 6B). Thus, it appears that the inhibitory action of CTHRC1 occurs at the top of the adipogenic transcription cascade. To test this, the effect of CTHRC1 on CREB Ser¹³⁵ activating phosphorylation induced by adipogenic differentiation is assessed. In addition, it is determined whether CTHRC1 inhibits early (Thr¹⁸⁸) and late (Thr¹⁷⁹ or Ser¹⁸⁴) phosphorylation of C/EBPβ. When in vitro studies identify the full spectrum of adipogenic transcription factors regulated by CTHRC1, these results are verified using the in vivo models developed above. Western blotting and qRT-PCR is used to assess the activating phosphorylation and expression of adipogenic transcription factors in subcutaneous and visceral adipose depots of original Cthrc1 null mice and mice with inducible liver-specific expression of Cthrc1, with and without tamoxifen treatment. Recently it was found (FIG. 6G) that CTHRC1 prevents down regulation of the anti-adipogenic transcription regulator CHOP³⁴ normally observed after the induction of adipogenic differentiation. The role of CHOP in the anti-adipogenic effect of CTHRC1 is examined using RNAi approaches. The effects of CTHRC1 on adipogenesis are compared between the preadipocytes expressing CHOP shRNA and control scrambled shRNA. In addition, the expression of CHOP in the white adipose tissue of Cthrc1 null, Cthrc1 transgenic and control mice is analyzed.

### Does CTHRC1 Affect Enzymes Participating in Lipid Biosynthesis?

Adipogenesis culminates in a dramatic enhancement of triglyceride production, which is dependent on lipogenesis, conversion of acetyl-CoA to fatty acids. Two enzymes are playing key roles in lipogenesis, pyruvate dehydrogenase (PDH) converting pyruvate to acetyl-CoA, and Acetyl-CoA carboxylase (ACC) responsible for conversion of acetyl CoA to malonyl-CoA. Both enzymes are activated by specific dephosphorylation. Immunoblotting with commercial antibodies is applied against total and phosphorylated PDH and ACC to test whether CTHRC1 affects their phosphorylation in nondifferentiated and differentiated preadipocytes. In addition, qRT-PCR is used to test whether CTHRC1 regulates expression of ACC, PDH, and PDH phosphatase. Because CTHRC1 decreases PPARγ activity, qRT-PCR and immunoblotting are used to evaluate the effect of CTHRC1 treatment on the expression of lipogenesis-related genes regulated by PPARγ, such as adipocyte fatty acid binding protein, lipoprotein lipase, GLUT4 and fatty acid transport protein. These in vitro experiments are followed by in vivo studies testing the effects of Cthrc1 induction on the expression of genes involved in lipid biosynthesis. Also, an innovative unbiased lipidomics approach by mass spectrometry²³ is used to identify changes in lipid composition in adipose depots in mouse models (Table 3). Preliminary studies have already been extremely informative in defining classes of lipids as well as unique features of specific adipose depots within the body.

TABLE 3
Mass-spectrometry reveals dramatic qualitative and quantitative differences
in lipid species from Cthrc1 null (KO) and wildtype (WT) mice.

| | Lipid class | | | | | |
|---|---|---|---|---|---|---|
| | TAGs, SMs, DAGs, MADAGs; | | | | | |
| | KO vs. WT | | | GPLs, KO vs. WT | | |
| | N, | | dominant | | | dominant |
| Fat depot | p < 0.05 | range | constituent | N, p < 0.05 | range | constituent |
| Inguinal | 13 | >10⁴ | TAGs, SM (up) | 72 | <30 | PE, PC (down) |
| Retroperitoneal | 160 | >2.1 × 10⁴ | TAGs (up) | 60 | <40 | PE, PC (down) |

In Table 3, range is overall fold difference. TAGs-triacylglycerides, DAGs-diacylglycerides, SMs-sphingomyelins, GPLs-glycerophospholipids, MADAGs-monoalkyldiacylglycerols Similar analyses are conducted with adipose depots from Cthrc1 overexpressing mice as recently published²³. Software used for analyses included LipidView, PeakView, and MarkerView.

### Does CTHRC1 Affect the Proliferation of Mitochondria and Enhancement of Oxidative Phosphorylation in the Course of Adipogenic Differentiation?

Adipogenesis is accompanied by a strong increase of mitochondrial abundance and overall enhancement of oxidative phosphorylation (OXPHOS), and knockdown of mitochondrial transcription factor A strongly inhibits adipogenesis³⁵. Considering the importance of OXPHOS for adipogenesis, it is proposed to assess the effect of CTHRC1 on the mitochondrial apparatus and mitochondrial respiration. First the effect of CTHRC1 treatment and/or CTHRC1 overexpression in Cthrc1 null preadipocyte cultures on the ensemble of mitochondria is elucidated. To this end, live cells before and after the induction of adipogenic differentiation, in presence or absence of CTHRC1, are stained with Mitotracker. Then, mitochondria are visualized by three-dimensional confocal reconstruction and their summary volume determined using Imaris software. In addition, to characterize mitochondrial activity, the effect of CTHRC1 on mitochondrial membrane potential are determined by supravital confocal microscopy of live cells stained with a Rhodamine 123 probe. Next, the effect of CTHRC1 on OXPHOS and glycolysis is elucidated using the Seahorse XF Analyzer, based respectively on oxygen consumption and extracellular acidification rates. In case CTHRC1 suppresses the proliferation of mitochondria and overall OXPHOS increase in the cells induced to differentiate CTHRC1's effect on the expression of mitochondrial enzymes involved in OXPHOS, such as enzymes of the electron transport chain and ATP synthase, and in mitochondrial growth, such as DNA polymerase k, TWINKLE and mitochondrial SSB proteins is determined.

### Why is Brown Adipogenesis Refractory to CTHRC1?

The finding that CTHRC1 does not affect differentiation of immortalized brown preadipocytes (FIG. 6E and FIG. 6F) indicates that signaling pathways determining white and brown adipogenesis are differentially regulated by CTHRC1. The resistance of brown adipogenesis to CTHRC1 in conjunction with the suppression of white adipogenesis could enhance the overall beneficial effect of CTHRC1 on the metabolic status of the organism. Considering the importance of this finding, it is proposed to elucidate the effects of CTHRC1 on the expression of protein markers and transcriptional determinants of brown adipogenesis. To induce differentiation, immortalized BAT cells are incubated for 2 days with a cocktail of insulin, IBMX, indomethacin, dexamethasone and T3 hormone followed by 2-3 days with insulin and T3 only. First, qRT-PCR and immunoblotting are used to compare markers of BAT, Ucp1 and Kcnk3³⁶ in CTHRC1-treated and control BAT cells at different stages of chemically induced differentiation. Next, the effect of CTHRC1 on the brown adipocyte transcription factor PRDM16 and common adipogenesis regulators C/EBPβ, C/EBPδ and C/EBPα and anti-adipogenic regulator CHOP is analyzed. In addition, the effect of CTHRC1 on the activating phosphorylation of CREB and C/EBPβ in differentiating BAT cells is determined using immunoblotting. Following these studies, expression of brown adipogenesis regulators in BAT and WAT is assessed in transgenic mice after induction of the Cthrc1 transgene.

### Assess Binding of CTHRC1 and GPR180 and CTHRC1-Induced GPR180 Internalization

The data suggest GPR180 is a CTHRC1 receptor, and this interaction is further characterized herein. Stimulation with ligands usually induces the internalization of ligand/GPR complexes³⁷. Fluorescence resonance energy transfer (FRET) enables detection of direct binding between proteins in situ³⁸. To apply FRET to CTHRC1/GPR180 binding, CHO-K1 cells transfected with the Gpr180 expression construct are used. Transfected cells are incubated at 4° C. with CTHRC1 and then formalin fixed. To detect CTHRC1, the rabbit monoclonal antibody Vli55 is labeled with FITC using the Invitrogen FluoReporter kit. For detection of GPR180, the specific mouse antibody in hand is labeled with Cy3 (anti-myc tag monoclonal Vli1 used on GPR180-myc transfected cells as alternative). FRET studies will be performed using the Leica SP8 confocal microscope and acceptor bleaching method. A significant increase of green fluorescence after specific bleaching of Cy3 will reflect FRET and indicate a direct interaction between CTHRC1 and GPR180. The activation of G protein-coupled receptors leads to their internalization in endosomes that after maturation can fuse with lysosomes³⁷. To assess the ability of CTHRC1 to induce the internalization of GPR180, CHO-K1 cells transfected with Gpr180 are used. In the first experiments, cells are incubated with CTHRC1-containing conditioned medium for 2, 5, 15 or 30 min followed by formalin fixation. Cells are co-stained with CY3-conjugated mouse anti-GPR180 antibodies, and rabbit anti-clathrin (endosome marker) or anti-LAMP2 (lysosome marker) antibodies followed by secondary Alexa 488-conjugated antibodies. Membranes are stained with the far-red lipophilic dye Deep Red (Molecular Probes). Confocal microscopy is used to localize GPR180 vis-à-vis endosomes and lysosomes at different time points after CTHRC1 stimulation.

### Assess the Activation of GPR180 by CTHRC1

It is verified that binding of CTHRC1 activates GPR180 using the PathHunter β-Arrestin GPCR assay (DiscoverX). This cell-based functional assay directly measures GPCR activity by detecting interaction of β-Arrestin with the activated GPCR, independent of G-protein signaling. Gpr180 is cloned in frame with the β-gal enzyme fragment (ProLink, enzyme complementation assay). This construct is transfected into CHO cells stably expressing β-Arrestin and the enzyme acceptor deletion mutant of the β-gal enzyme. Upon activation of GPR180, binding of β-Arrestin to the ProLink-tagged GPCR occurs, which forces complementation of the two enzyme fragments and formation of active β-gal, which will be measured using chemiluminescent detection reagents.

### Characterize the Role of GPR180 in the Anti-Adipogenic Effect of CTHRC1

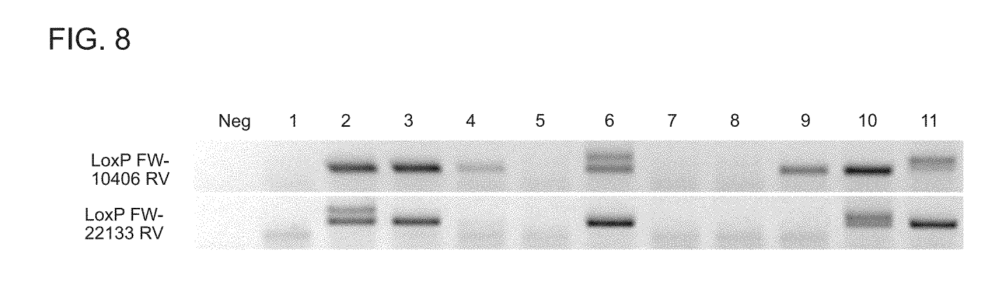

3T3-L1 cells are transduced with retroviral constructs for Gpr180 shRNA (Origene), and Gpr180 suppression is verified by qRT-PCR and immunoblotting. The effect of CTHRC1 on adipogenic differentiation in Gpr180 shRNA and scrambled shRNA transfected cells is compared using Oil red 0 staining and perilipin immunoblotting. In addition, Gpr180 shRNA transfectants is used to elucidate whether Gpr180 knockdown rescues PPARγ expression after CTHRC1 treatment. These results are verified in later studies using the Gpr180 null mice successfully generated with CRISPR/Cas9 technology (FIG. 8).

### Identify Signaling Pathways Activated Downstream of GPR180 in Response to CTHRC1

These studies will determine the signaling response to ligand-stimulated GPR180. Ten (10) common pathways are examined using the GPCR Signaling Finder Reporter Array (Qiagen). This is a cell based reporter assay that measures the functional consequences of GPCR activation or inhibition. Among the common pathways are cAMP/PKA (CREB reporter), calcium/PKC (NFAT reporter), ERK and JNK (ELK1/SRF and FOS/JUN reporter), PI-3 kinase/AKT (FOXO reporter), MEF2, hedgehog (GLI reporter), NF-κB, and JAK/STAT (STATS reporter). Using these constructs, dual-luciferase assays is performed on CHO cells stably transduced with a Gpr180 lentiviral vector and stimulated with CTHRC1. This approach identifies the specific signaling pathways affected by GPR180 signaling. It has been reported that CTHRC1 inhibits CREB reporter activity in differentiating 3T3-L1 cells³. Future small molecule screens for GPR180 agonists and antagonists will build on this information to establish screening assays. Signaling through G_{αs} and G_{αi/o} regulates the activity of adenylate cyclase and accumulation of cyclic adenosine monophosphate (cAMP)³⁹. Signaling through G_{αq/11} activates phospholipase C-β and results in increase of intracellular Ca²⁺ concentration⁴⁰. Finally, G_{α12/13} pathway activates the actin cytoskeleton regulator Rho GTPase⁴¹. In addition to use of reporter constructs, confocal fluorescence methods are used to detect pathway activation in response to CTHRC1 binding to GPR180. Confocal studies are performed on live GPR180-transfected cells. To assess the level of cAMP in CTHRC1-stimulated cells, cell transfection with the Epac1-camps FRET sensor is utilized. To assess the effect of GPR180 activation on the intracellular Ca²⁺ concentration, the Calcium Green-1 AM cell permeable fluorescent calcium sensor (Molecular Probes) is applied. Because the available fluorescent sensors of Rho activation are not yet sufficiently reliable, a commercial pull-down kit (Enzo) is used to assess Rho activity in the extracts of Gpr180 transfected CHO-K1 cells after treatment with CTHRC1. In addition, confocal microscopy is used to analyze abundance of F-actin stress fibers, which is enhanced by activated Rho. CHO-K1 cells are cotransfected with Gpr180 and an available actin-GFP construct. Confocal image stacks (excitation 488 nm, emission 500-530 nm) are taken at different time points after cell stimulation with CTHRC1. 3D reconstruction of the images and determination of stress fiber density are performed. If the stimulation of stress fiber formation by CTHRC1 is observed, it is examined whether it is sensitive to Rho inhibitor Y-27362.

### Expected Results, Potential Problems and Alternative Approaches

It is expected that these experiments will result in: (i) identification of pro-adipogenic transcription factors that are under direct or indirect control of Cthrc1 signaling; (ii) understanding whether lipid biosynthesis enzymes and status of the mitochondrial apparatus are affected by Cthrc1 signaling; (iii) understanding the mechanism of brown adipogenesis resistance to CTHRC1. The significance of these data will be assessed in the experiments involving inducible overexpression or knockdown of specific genes regulated by CTHRC1 and determination of their role in the anti-adipogenic effect of CTHRC1. Taken together, the results will facilitate the construction of a comprehensive scheme of adipogenesis control by CTHRC1. The methods to be used in the proposed studies are state-of-the art, and major technical problems are not expected. Cthrc1 null mice characteristically develop liver steatosis at several months of age. If time permits, it will be assessed whether the molecular mechanisms of adipogenesis regulation by CTHRC1 also apply to steatosis. The recently described model of induced in vitro steatosis in rodent hepatocytes is used⁴². If, CTHRC1 inhibits in vitro steatosis, similar analyses will follow as described above for preadipocytes.

In addition, it is expected that experiments above will characterize the interaction between CTHRC1 and GPR180 and the downstream signaling events. In the unlikely case of problems with FRET, the commercial Duolink system for the study of GPR180 and CTHRC1 interactions in situ is an alternative. It is expected that at least one major pathway downstream of G protein-coupled receptors will be activated by CTHRC1 in cells expressing GPR180. As a potential alternative approach, CHO suspension cells may be used for flow cytometry studies of cAMP and Ca²⁺ accumulation using respectively Epac1-camps and Fura-2. Identification of signaling pathways(s) activated by CTHRC1 will be critical for detailed understanding of signaling underlying the regulation of adipogenic transcription factors by CTHRC1. As an alternative to the use of GPCR Signaling Finder Reporter Array, unbiased phosphoproteomics may be applied to identify the proteins phosphorylated after CTHRC1 application. If results above validate the signaling of CTHRC1 through GPR180, genetic in vivo studies will confirm this to complement the studies above. CRISPR/Cas technology was used to create a global and conditional null allele of Gpr180. The global knockout has been successfully generated, and mice are characterized where loxP sites were targeted to flank exons 3 and 6 (FIG. 8). These mice were individually bred, and genotyping of the F1 mice confirmed that there were 7 mice with a foxed Gpr180 allele and 3 global null mice. This allele will be backcrossed to eliminate potential off target integration of the loxP sites. High titer polyclonal mouse anti-GPR180 antibody suitable for Western blotting and immunohistochemistry have been generated herein. The antibodies will be used to verify absence of the GPR180 protein by Western blotting and immunostaining of all organs. Studies will focus on the most prominent abnormalities observed in Cthrc1 null mice and interrogate whether similar pathology occurs in Gpr180 mutants. The second potential strategy with the conditional Gpr180 mutant mice is to use Slc17a6-Cre to delete Gpr180 from the brain to further test effects of neuronal loss of sensitivity to local CTHRC1. This experiment is also important if prior studies determine that circulating CTHRC1 can pass through the blood brain barrier and act on neuronal cells. Collectively, these results are highly impactful because an entirely field with implications for obesity, physical activity and muscle performance is essentially being established. Of further interest is the fact that these pathways are signaling targets to address a critical health problem. With so many unanswered questions about the functions of CTHRC1, the examples herein prioritize and focus on aspects most compatible with expertise. The CTHRC1-GPR180 signaling system is an attractive therapeutic target because neither loss of Cthrc1 nor loss of Gpr180 impairs viability during development. Furthermore, G protein-coupled receptors are ideal targets for drug development.

### Example 6: Confirmation of Activation of CTHRC1 Via a Nutritional, Trans-Generational Effect and Determination Whether this Activation Affects Body Composition in Offspring

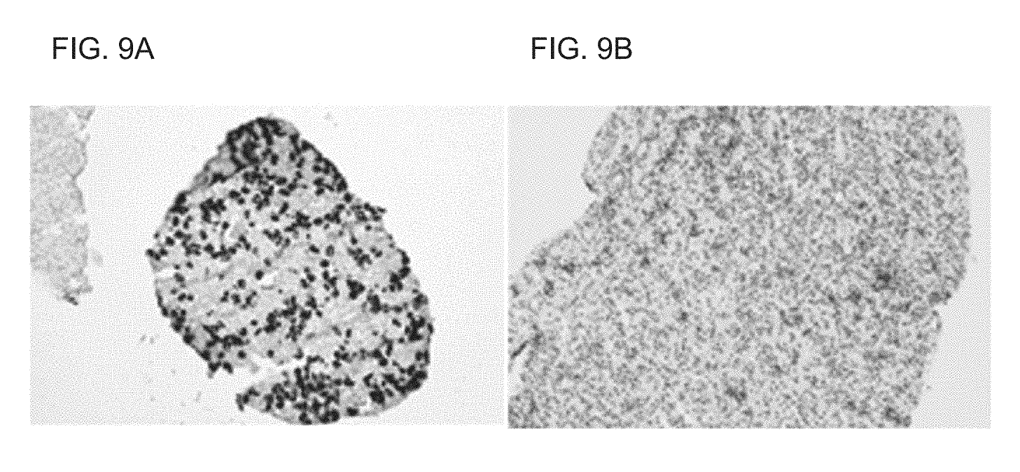

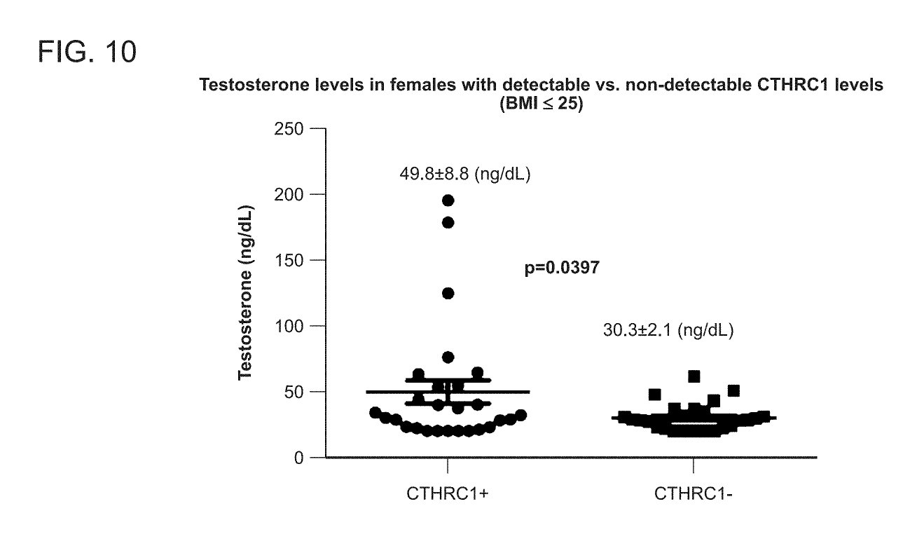

As described herein, observations points to potential nutritional, trans-generational regulation of CTHRC1 expression, which could potentially be epigenetic regulation. Pituitary glands from rats from a protein malnutrition study were received^{43,44}. Rats were bred while on a 6% (malnutrition) or 25% (normal) protein diet, which was maintained throughout pregnancy. Immediately after birth, 8 pups from each offspring were transferred to foster mothers being fed standard 25% protein content diet, and pituitary glands were harvested after weaning for CTHRC1 immunostaining (FIG. 9). The pituitary gland from the rat experiencing protein malnutrition conditions during development had high levels of CTHRC1 protein, whereas none was detected in the control pituitary from offspring of a mother fed standard chow unrestricted. Examples of epigenetic regulation of IGF2 expression levels in humans has been demonstrated in subjects exposed to prenatal famine⁴⁵. These preliminary data are particularly intriguing given the fact that in humans increased gestational weight gain or intrauterine exposure to an obesity-related environment determines obesity risk in the offspring by influencing appetite, metabolism, and activity levels^{46,47}. In the context of data that CTHRC1 is involved in regulating physical activity and inhibition of adipogenesis³, the fact that CTHRC1 levels are detectable only in approximately 20% of the human population raises the pressing question of how CTHRC1 levels are regulated. The significance of this question cannot be overstated when the preliminary data that champion endurance athletes typically have very high CTHRC1 levels and low body fat are considered (FIG. 2). Preliminary data suggest that exercise per se does not influence CTHRC1 levels and CTHRC1 levels in Ironman competitors were similar before and after the event (FIG. 2). Understanding how CTHRC1 level are determined could therefore potentially translate readily into effective ways to prevent obesity. Therefore, the relationship of gestational nutritional status and CTHRC1 levels in offspring is assessed.

As described herein, hormonal CTHRC1 levels are increased in the offspring of nutritionally-deprived mothers.

### Does Prenatal Protein-Malnutrition Increase Circulating Levels of CTHRC1 and CTHRC1 in the Pituitary?

Based on the finding of CTHRC1 in a rat pituitary, the proposed studies are conducted in Long-Evans rats using a similar protocol as described^{43,44}. Females are placed on a 6% protein restricted diet (Teklad TD.90016) 5 weeks before mating with males placed on the low protein diet 1 week before mating. Control matings receive standard breeder chow (Teklad 2919 Global) throughout the study. At birth, blood is obtained from parents and pups for CTHRC1 ELISA, and pituitary glands and all major organs is collected for CTHRC1 expression analysis by immunohistochemistry, Western blotting, and qRT-PCR (n=8 newborn males and n=8 newborn females per group). In a separate set of animals, the same dietary scheme is implemented; however, after birth 16 newborns from each litter are transferred to foster mothers that were always fed breeder chow. CTHRC1 plasma levels and body weight/body composition (NMR) is determined weekly after birth. At 90 days of age (P90, n=16 rats per group, male and female) all rats are euthanized for analysis of tissues as described for newborns, including quantification of adipose depots. This experiment examines whether CTHRC1 expression levels remain elevated despite reversal of the protein malnutrition status, which would be indicative of epigenetic regulation of Cthrc1 gene expression. It is predicted that maternal malnutrition will yield offspring with high levels of pituitary CTHRC1 expression resulting in increased circulating CTHRC1 levels. It is also anticipated that gestational malnutrition will result in reduced birth weight that normalizes over time^{43,44}.

### Does Postnatal Protein-Malnutrition Increase Circulating Levels of CTHRC1 and CTHRC1 Expression in the Pituitary?

This experiment addresses whether CTHRC1 levels are influenced by nutritional status after weaning. At 8 weeks of age groups of rats (n=16, male and female) born and raised on normal chow are randomized to receive a 6% protein diet or normal chow for 5 weeks. Body weight and composition is determined weekly along with CTHRC1 plasma levels during the dietary intervention. Analyses of tissues are carried out as described above. If CTHRC1 levels are sensitive to nutritional cues during adulthood, a follow-up experiment will be conducted that will ask whether CTHRC1 levels raised in adults in response to nutritional restriction will decrease again when normal chow is introduced.

### Expected Results, Potential Problems, and Alternative Approaches

It is anticipated that CTHRC1 expression in the anterior pituitary will turn out to be epigenetically regulated. It is expected that gestational malnutrition-induced CTHRC1 expression remains elevated into adulthood. Similar to most human subjects, it was determined that CTHRC1 levels in wildtype inbred mice born and raised on normal chow are below detectable levels. It is also determined whether caloric restriction instead of protein malnutrition has similar effects on the regulation of CTHRC1 levels. The underlying mechanism of a potential epigenetic regulation of pituitary CTHRC1 expression is also investigated. Within 140 bp upstream of the Cthrc1 transcriptional start site are 13 CpG sites that could be subject to methylation for control of transcription. The potential influence of histone modifications and miRNAs is also examined. The results of these studies could provide an explanation and a justification for controlling gestational weight gain to prevent obesity in humans in the future. An association of high birth weight (>4000 g) in humans with development of subsequent obesity has been confirmed⁴⁸. Also, effects of malnutrition during pregnancy can vary among species, even between rats and mice⁴⁹⁻⁵¹.

### REFERENCES CITED

1. Ogden C L, Carroll M D, Kit B K and Flegal K M. Prevalence of childhood and adult obesity in the United States, 2011-2012. JAMA. 2014; 311:806-14.
2. Stohn J P, Perreault N G, Wang Q, Liaw L and Lindner V. Cthrc1, a novel circulating hormone regulating metabolism. PLoS One. 2012; 7:e47142.
3. Stohn J P, Wang Q, Siviski M E, Kennedy K, Jin Y R, Kacer D, DeMambro V, Liaw L, Vary C P, Rosen C J, Prudovsky I and Lindner V. Cthrc1 controls adipose tissue formation, body composition, and physical activity. Obesity (Silver Spring). 2015; 23:1633-1642.
4. Pyagay P, Heroult M, Wang Q, Lehnert W, Belden J, Liaw L, Friesel R E and Lindner V. Collagen triple helix repeat containing 1, a novel secreted protein in injured and diseased arteries, inhibits collagen expression and promotes cell migration. Circ Res. 2005; 96:261-8.
5. Ng S W and Popkin B M. Time use and physical activity: a shift away from movement across the globe. Obes Rev. 2012; 13:659-80.
6. Waalen J. The genetics of human obesity. Transl Res. 2014; 164:293-301.
7. Kushner R F and Ryan D H. Assessment and lifestyle management of patients with obesity: clinical recommendations from systematic reviews. JAMA. 2014; 312:943-52.
8. Ard J. Obesity in the US: what is the best role for primary care? BMJ. 2015; 350:g7846.
9. Robinson M K. Surgical treatment of obesity—weighing the facts. N Engl J Med. 2009; 361:520-1.
10. Joo J K and Lee K S. Pharmacotherapy for obesity. J Menopausal Med. 2014; 20:90-6.
11. Guzman A K, Ding M, Xie Y and Martin K A. Pharmacogenetics of obesity drug therapy. Curr Mol Med. 2014; 14:891-908.
12. Duarte C W, Stohn J P, Wang Q, Emery I F, Prueser A and Lindner V. Elevated plasma levels of the pituitary hormone cthrcl in individuals with red hair but not in patients with solid tumors. PLoS One. 2014; 9:e100449.
13. Schutz U H, Schmidt-Trucksass A, Knechtle B, Machann J, Wiedelbach H, Ehrhardt M, Freund W, Groninger S, Brunner H, Schulze I, Brambs H J and Billich C. The TransEurope FootRace Project: longitudinal data acquisition in a cluster randomized mobile MRI observational cohort study on 44 endurance runners at a 64-stage 4,486 km transcontinental ultramarathon. BMC Med. 2012; 10:78.
14. Knechtle B, Wirth A, Alexander Rust C and Rosemann T. The Relationship between Anthropometry and Split Performance in Recreational Male Ironman Triathletes. Asian J Sports Med. 2011; 2:23-30.
15. Rust C A, Knechtle B, Knechtle P, Wirth A and Rosemann T. A comparison of anthropometric and training characteristics among recreational male Ironman triathletes and ultra-endurance cyclists. Chin J Physiol. 2012; 55:114-24.
16. Hoffman M D, Lebus D K, Ganong A C, Casazza G A and Van Loan M. Body composition of 161-km ultramarathoners. Int J Sports Med. 2010; 31:106-9.
17. Schuler M, Dierich A, Chambon P and Metzger D. Efficient temporally controlled targeted somatic mutagenesis in hepatocytes of the mouse. Genesis. 2004; 39:167-72.
18. Journals unite for reproducibility. Nature. 2014; 515:7.
19. McNutt M. Journals unite for reproducibility. Science. 2014; 346:679.
20. Clayton J A and Collins F S. Policy: NIH to balance sex in cell and animal studies. Nature. 2014; 509:282-3.
21. McCullough L D, de Vries G J, Miller V M, Becker J B, Sandberg K and McCarthy M M. NIH initiative to balance sex of animals in preclinical studies: generative questions to guide policy, implementation, and metrics. Biol Sex Differ. 2014; 5:15.
22. Hallakou S, Doare L, Foufelle F, Kergoat M, Guerre-Millo M, Berthault M F, Dugail I, Morin J, Auwerx J and Ferre P. Pioglitazone induces in vivo adipocyte differentiation in the obese Zucker fa/fa rat. Diabetes. 1997; 46:1393-9.
23. Liaw L, Prudovsky I, Koza R A, Anunciado-Koza R V, Siviski M E, Lindner V, Friesel R E, Rosen C J, Baker P R, Simons B and Vary C P. Lipid Profiling of In Vitro Cell Models of Adipogenic Differentiation: Relationships With Mouse Adipose Tissues. J Cell Biochem. 2016; 117:2182-93.
24. Spalding K L, Amer E, Westermark P O, Bernard S, Buchholz B A, Bergmann O, Blomqvist L, Hoffstedt J, Naslund E, Britton T, Concha H, Hassan M, Ryden M, Frisen J and Amer P. Dynamics of fat cell turnover in humans. Nature. 2008; 453:783-7.
25. Arch J R, Hislop D, Wang S J and Speakman J R. Some mathematical and technical issues in the measurement and interpretation of open-circuit indirect calorimetry in small animals. Int J Obes (Lond). 2006; 30:1322-31.
26. Kaiyala K J, Morton G J, Leroux B G, Ogimoto K, Wisse B and Schwartz M W.

Identification of body fat mass as a major determinant of metabolic rate in mice. Diabetes. 2010; 59:1657-66.
27. Kaiyala K J and Schwartz M W. Toward a more complete (and less controversial) understanding of energy expenditure and its role in obesity pathogenesis. Diabetes. 2011; 60:17-23.
28. Cawthorn W P, Scheller E L and MacDougald O A. Adipose tissue stem cells meet preadipocyte commitment: going back to the future. J Lipid Res. 2012; 53:227-46.
29. Reichart D L, Hinkle R T, Lefever F R, Dolan E T, Dietrich J A, Sibley D R and Isfort R J. Activation of the dopamine 1 and dopamine 5 receptors increase skeletal muscle mass and force production under non-atrophying and atrophying conditions. BMC Musculoskelet Disord. 2011; 12:27.
30. Zhang Y, Scislowski P W, Prevelige R, Phaneuf S and Cincotta A H. Bromocriptine/SKF38393 treatment ameliorates dyslipidemia in ob/ob mice. Metabolism. 1999; 48:1033-40.
31. Yamamoto S, Nishimura O, Misaki K, Nishita M, Minami Y, Yonemura S, Tarui H and Sasaki H. Cthrc1 selectively activates the planar cell polarity pathway of Wnt signaling by stabilizing the Wnt-receptor complex. Dev Cell. 2008; 15:23-36.
32. Tsukada S, Iwai M, Nishiu J, Itoh M, Tomoike H, Horiuchi M, Nakamura Y and Tanaka T. Inhibition of experimental intimal thickening in mice lacking a novel G-protein-coupled receptor. Circulation. 2003; 107:313-9.
33. Tang Q Q and Lane M D. Adipogenesis: from stem cell to adipocyte. Annu Rev Biochem. 2012; 81:715-36.
34. Batchvarova N, Wang X Z and Ron D. Inhibition of adipogenesis by the stress-induced protein CHOP (Gadd153). EMBO J. 1995; 14:4654-61.
35. Zhang Y, Marsboom G, Toth P T and Rehman J. Mitochondrial respiration regulates adipogenic differentiation of human mesenchymal stem cells. PLoS One. 2013; 8:e77077.
36. Svensson P A, Jernas M, Sjoholm K, Hoffmann J M, Nilsson B E, Hansson M and Carlsson L M. Gene expression in human brown adipose tissue. Int J Mol Med. 2011; 27:227-32.
37. Borroni E M, Mantovani A, Locati M and Bonecchi R. Chemokine receptors intracellular trafficking. Pharmacol Ther. 2010; 127:1-8.
38. Arai Y and Nagai T. Extensive use of FRET in biological imaging. Microscopy. 2013; 62:419-28.
39. Watts V J. Molecular mechanisms for heterologous sensitization of adenylate cyclase. J Pharmacol Exp Ther. 2002; 302:1-7.
40. McCudden C R, Haim M D, Kimple R J, Siderovski D P and Willard F S. G-protein signaling: back to the future. Cell Mol Life Sci. 2005; 62:551-77.
41. Siehler S. G12/13-dependent signaling of G-protein-coupled receptors: disease context and impact on drug discovery. Expert opinion on drug discovery. 2007; 2:1591-604.
42. Nativ N I, Yarmush G, Chen A, Dong D, Henry S D, Guarrera J V, Klein K M, Maguire T, Schloss R, Berthiaume F and Yarmush M L. Rat hepatocyte culture model of macrosteatosis: effect of macrosteatosis induction and reversal on viability and liver-specific function. J Hepatol. 2013; 59:1307-14.
43. Amaral A C, Jakovcevski M, McGaughy J A, Calderwood S K, Mokler D J, Rushmore R J, Galler J R, Akbarian S A and Rosene D L. Prenatal protein malnutrition decreases KCNJ3 and 2D G activity in rat prefrontal cortex. Neuroscience. 2015; 286:79-86.
44. Mokler D J, Torres O I, Galler J R and Morgane P J. Stress-induced changes in extracellular dopamine and serotonin in the medial prefrontal cortex and dorsal hippocampus of prenatally malnourished rats. Brain Res. 2007; 1148:226-33.
45. Heijmans B T, Tobi E W, Stein A D, Putter H, Blauw G J, Susser E S, Slagboom P E and Lumey L H. Persistent epigenetic differences associated with prenatal exposure to famine in humans. Proc Natl Acad Sci USA. 2008; 105:17046-9.
46. Oken E. Maternal and child obesity: the causal link. Obstet Gynecol Clin North Am. 2009; 36:361-77, ix-x.
47. Oken E, Kleinman K P, Belfort M B, Hammitt J K and Gillman M W. Associations of gestational weight gain with short- and longer-term maternal and child health outcomes. Am J Epidemiol. 2009; 170:173-80.
48. Yu Z B, Han S P, Zhu G Z, Zhu C, Wang X J, Cao X G and Guo X R. Birth weight and subsequent risk of obesity: a systematic review and meta-analysis. Obes Rev. 2011; 12:525-42.
49. Cripps R L, Martin-Gronert M S, Archer Z A, Hales C N, Mercer J G and Ozanne S E. Programming of hypothalamic neuropeptide gene expression in rats by maternal dietary protein content during pregnancy and lactation. Clin Sci (Lond). 2009; 117:85-93.
50. Bellinger L, Sculley D V and Langley-Evans S C. Exposure to undernutrition in fetal life determines fat distribution, locomotor activity and food intake in ageing rats. Int J Obes (Lond). 2006; 30:729-38.
51. Ravelli G P, Stein Z A and Susser M W. Obesity in young men after famine exposure in utero and early infancy. N Engl J Med. 1976; 295:349-53.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of predicting endurance in a subject, the method comprising:
providing a test sample from the subject;
determining a level of Collagen Triple Helix Repeat Containing 1 (CTHRC1) in the test sample from the subject;
comparing the level of CTHRC1 in the subject to a control level, wherein a higher level of CTHRC1 in the test sample relative to the control sample indicates that the subject has high endurance.

2. The method of claim 1, wherein the level of CTHRC1 is determined using an enzyme-linked immunosorbent assay (ELISA).

3. The method of claim 2, wherein the subject with high endurance has better physical performance under physically stressful conditions as compared to a subject with low or normal endurance.

4. The method of claim 1, wherein the test sample comprises a blood sample.

5. The method of claim 4, wherein the blood sample comprises a plasma sample.


SEQUENCE LISTING (3 sequences)

SEQ ID NO: 1 - protein, 243 aa, Homo sapiens [sequence omitted]

SEQ ID NO: 2 - DNA, 1279 bp, Homo sapiens
gggagggagagaggcgcgcgggtgaaaggcgcattgatgcagcctgcggcggcctcggagcgcggcggagccagacgctgaccacgttcctctcctcggtctcctccgcctccagctccgcgctgcccggcagccgggagccatgcgaccccagggccccgccgcctccccgcagcggctccgcggcctcctgctgctcctgctgctgcagctgcccgcgccgtcgagcgcctctgagatccccaaggggaagcaaaaggcgcagctccggcagagggaggtggtggacctgtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggtacttgttcagattacccaaaaggagatgcttctactggatggaattcagtttctcgcatcattattgaagaactaccaaaataaatgctttaattttcatttgctacctctttttttattatgccttggaatggttcacttaaatgacattttaaataagtttatgtatacatctgaatgaaaagcaaagctaaatatgtttacagaccaaagtgtgatttcacactgtttttaaatctagcattattcattttgcttcaatcaaaagtggtttcaatattttttttagttggttagaatactttcttcatagtcacattctctcaacctataatttggaatattgttgtggtcttttgttttttctcttagtatagcatttttaaaaaaatataaaagctaccaatctttgtacaatttgtaaatgttaagaattttttttatatctgttaaataaaaattatttccaacaaccttaatatctttaaa

SEQ ID NO: 3 - DNA, 1214 bp, Homo sapiens
agaaggtttaaggccggaaagggaaatgaaggggcccggcgctaaccctctaaggacctgttttgcttctgtttaaaccaaatgggcagtctgtcattacacacaccctgggtcttcatatgtggccgccaggtaggagcatcacagtcaagctacgggagaaaacagtttccaggaaactggaaatgaacggcccgagtgctttccaggggctcatctgtgggaagtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggtacttgttcagattacccaaaaggagatgcttctactggatggaattcagtttctcgcatcattattgaagaactaccaaaataaatgctttaattttcatttgctacctctttttttattatgccttggaatggttcacttaaatgacattttaaataagtttatgtatacatctgaatgaaaagcaaagctaaatatgtttacagaccaaagtgtgatttcacactgtttttaaatctagcattattcattttgcttcaatcaaaagtggtttcaatattttttttagttggttagaatactttcttcatagtcacattctctcaacctataatttggaatattgttgtggtcttttgttttttctcttagtatagcatttttaaaaaaatataaaagctaccaatctttgtacaatttgtaaatgttaagaattttttttatatctgttaaataaaaattatttccaacaaccttaatatctttaaa